(19) United States
(12) Patent Application Publication
(10) Pub. No.: US 2011/0081428 A1
(43) Pub. Date: Apr. 7, 2011

(54) Use of thioflavin-like compounds to increase life span and/or health span

(75) Inventors: Gordon J. Lithgow, Novato, CA (US); Silvestre de J. Alavez Espidio, Novato, CA (US)

(73) Assignee: The Buck Institute for Age Research

(21) Appl. No.: 12/807,938

(22) Filed: Sep. 16, 2010

Related U.S. Application Data
(60) Provisional application No. 61/276,892, filed on Sep. 16, 2009.

(51) Int. Cl. A61K 31/428; A61K 31/423; A61K 31/4184; A61K 31/436; A61K 31/421; A61K 33/00; A61P 19/10; A61P 19/02; A61P 27/12; A61P 27/02; A61P 9/00; A61P 3/06

(52) U.S. Cl. 424/722; 514/367; 514/375; 514/394; 514/291; 514/376

(57) ABSTRACT

The present invention provides a method of using thioflavin and functionally similar compounds to increase life span and/or health span.

### CROSS-REFERENCE TO RELATED APPLICATIONS

This application claims the benefit of U.S. provisional application No. 61/276,892, filed Sep. 16, 2009, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with government support under grant no. R01AG029631-01A 1 awarded by the National Institutes of Health. The Government has certain rights in the invention.

### FIELD OF THE INVENTION

In certain embodiments, this invention pertains to the use of thioflavin and functionally similar compounds to increase life span and/or health span and related pharmaceutical compositions.

### BACKGROUND OF THE INVENTION

Thioflavin T (4-(3,6-dimethyl-1,3-benzothiazol-3-ium-2-yl)-N,N-dimethylaniline chloride (ThT)) is a basic dye first described as a selective amyloid dye in 1959 by Vassar and Culling (Arch. Pathol. 68: 487 (1959)). Schwartz et al. (Zbl. Path. 106: 320 (1964)) first demonstrated the use of Thioflavin S, an acidic dye, as an amyloid dye in 1964. The properties of both Thioflavin T and Thioflavin S have since been studied in detail. Kelenyi J. Histochem. Cytochem. 15: 172 (1967); Burns et al. J. Path. Bact. 94:337 (1967); Guntern et al. Experientia 48: 8 (1992); LeVine Meth. Enzymol. 309: 274 (1999). Thioflavin S is commonly used in the post-mortem study of amyloid deposition in AD brain where it has been shown to be one of the most sensitive techniques for demonstrating senile plaques. Vallet et al. Acta Neuropathol. 83: 170 (1992). Thioflavin T has been frequently used as a reagent to study the aggregation of soluble amyloid proteins into beta-sheet fibrils. LeVine Prot. Sci. 2: 404 (1993). Quaternary amine derivatives related to Thioflavin T have been proposed as amyloid imaging agents, although no evidence of brain uptake of these agents has been presented. Caprathe et al., U.S. Pat. No. 6,001,331. Thioflavin compounds, methods of synthesizing the compounds, and methods of using the compounds in, for example, in vivo imaging of patients having neuritic plaques, and treatment of patients having diseases where accumulation of neuritic plaques are prevalent are described in U.S. Pat. No. 7,270,800.

### SUMMARY OF THE INVENTION

In certain embodiments, the invention provides a method of improving a measure of life span and/or health span. The method entails administering an effective amount of a thioflavin compound, or derivative thereof, to a subject, whereby the measure of life span and/or health span is improved, and wherein the thioflavin compound has one of structures A-E:

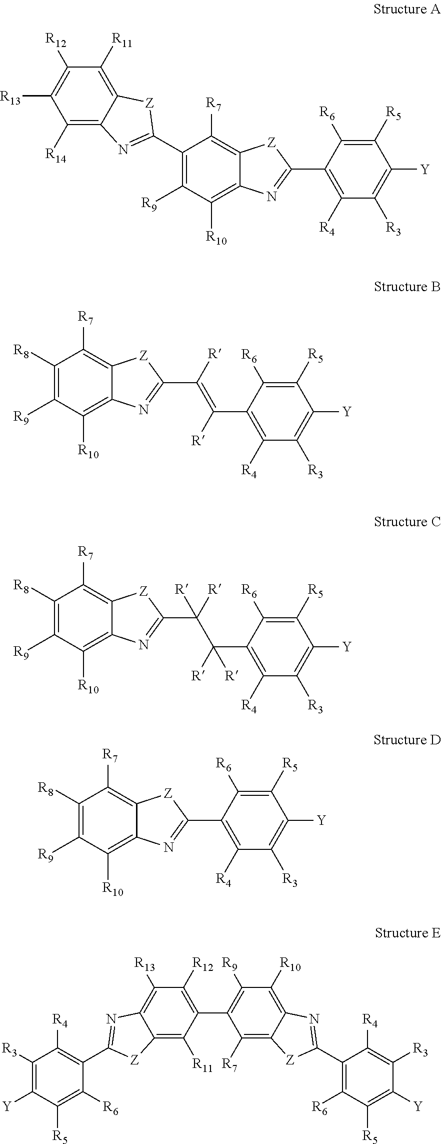

wherein Z is S, NR′, 0 or CR′ in which case the correct tautomeric form of the heterocyclic ring becomes an indole in which R′ is H or a lower alkyl group:

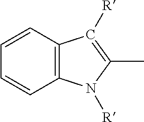

wherein Y is NR¹R², OR², or SR²;
wherein the nitrogen of

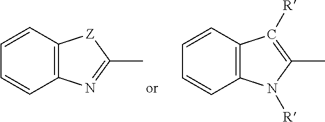

is not a quarternary amine; or an thioflavin compound having one of structures F-J or a water soluble, non-toxic salt thereof:

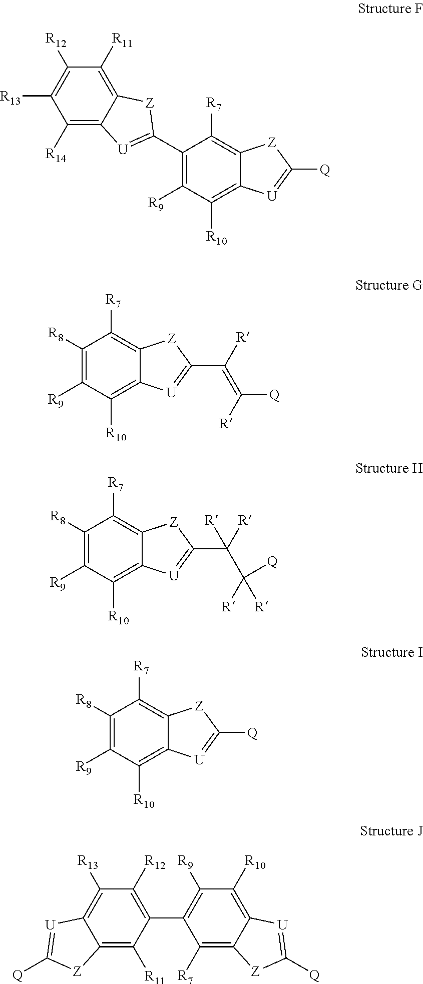

wherein each Q is independently selected from one of the following structures:

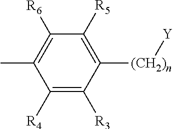

wherein n=0, 1, 2, 3 or 4,

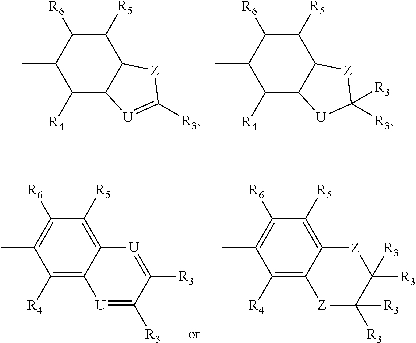

wherein Z is S, NR′, 0, or C(R′)₂ in which R′ is H or a lower alkyl group;
wherein U is CR′ (in which R′ is H or a lower alkyl group) or N (except when U═N, then Q is not

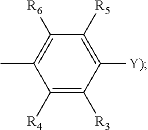

wherein Y is NR¹R², OR², or SR²;
wherein the nitrogen of

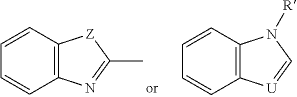

is not a quaternary amine;
wherein each R¹ and R² independently is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from any of the non-phenyl substituents defined below for R³-R¹⁴ and R′ is H or a lower alkyl group);
and wherein each R³-R¹⁴ independently is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, N0₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from any of the non-phenyl substituents defined for R¹-R¹⁴ and wherein R′ is H or a lower alkyl group), a tri-alkyl tin and a chelating group (with or without a chelated metal group) of the form W-L or V—W-L, wherein V is selected from the group consisting of: —COO—, —CO—, —CH₂O— and —CH₂NH—; W is —(CH₂)ₙ where n=0, 1, 2, 3, 4, or 5; and L is:

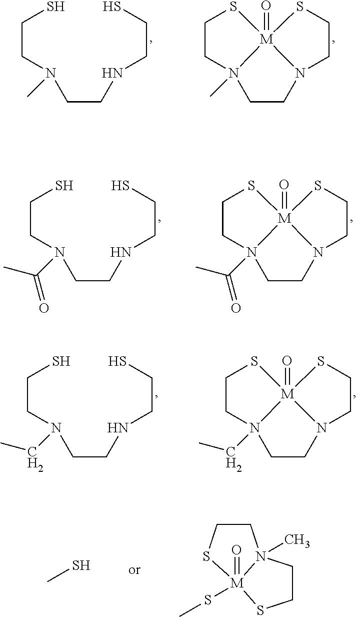

wherein M is selected from the group consisting of Tc, Re, Zn, Cu, Ni, V, Mn, Fe, Cr and Ru;
or wherein each R¹ and R² is a chelating group (with or without a chelated metal group) of the form W-L, wherein W is —(CH₂)ₙ where n=2, 3, 4, or 5; and L is:

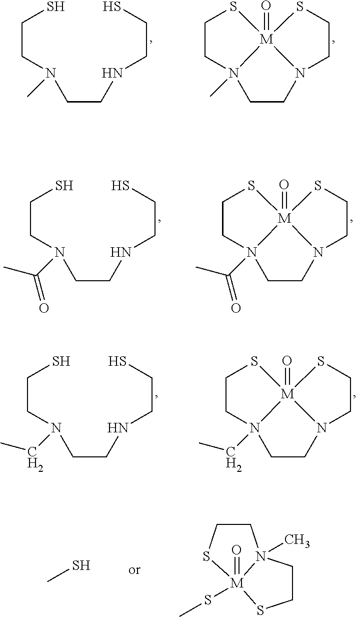

wherein M is selected from the group consisting of Tc and Re;
or wherein each R¹-R¹⁴ independently is selected from the group consisting of a chelating group (with or without a chelated metal ion) of the form W-L and V—W-L, wherein V is selected from the group consisting of —COO—, and —CO—; W is —(CH₂)ₙ where n=0, 1, 2, 3, 4, or 5; L is:

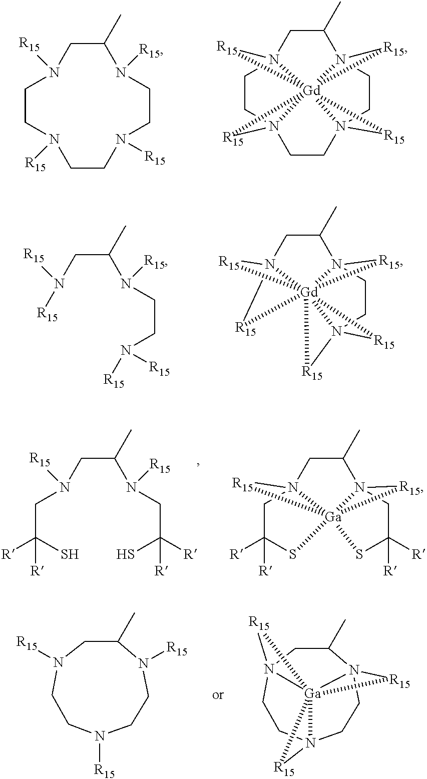

and wherein R¹⁵ independently is selected from the following:

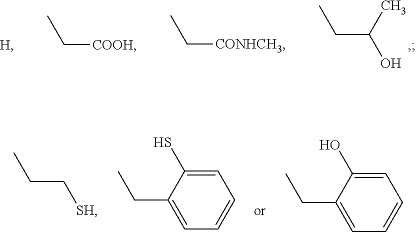

or a chelating compound (with or without a chelated metal group) or a water soluble, non-toxic salt thereof of the form:

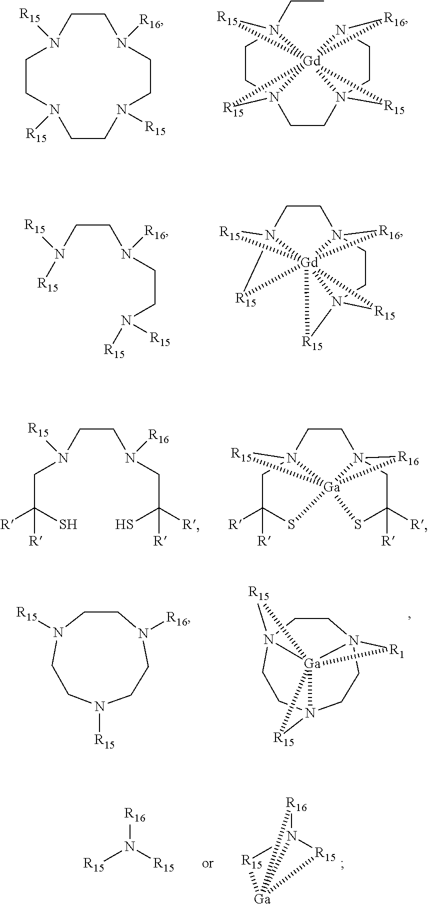

wherein R¹⁵ independently is selected from the following:

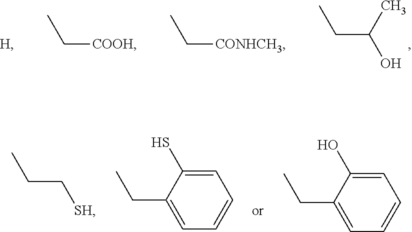

### and R¹⁶ is

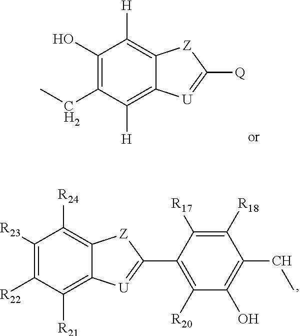

wherein
Q is independently selected from one of the following structures:

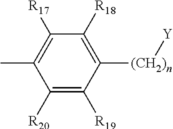

wherein n=0, 1, 2, 3 or 4,

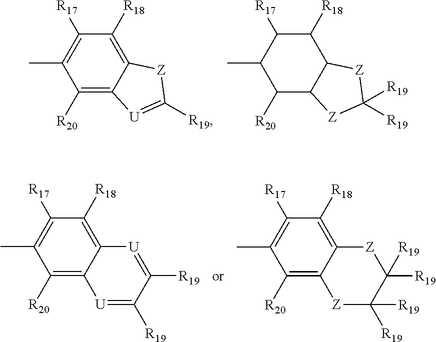

wherein Z is S, NR′, 0, or C(R)₂ in which R′ is H or a lower alkyl group;
wherein U is N or CR′;
wherein Y is NR¹R², OR², or SR²;
wherein each R¹⁷-R²⁴ independently is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ and CR₂′—CR₂′—Rₚₕ (wherein Rₚₕ represents an unsubstituted or substituted phenyl group with the phenyl substituents being chosen from any of the non-phenyl substituents defined for R¹⁷-R²⁰ and wherein R′ is H or a lower alkyl group).

In an illustrative embodiment, Thioflavin T (4-(3,6-dimethyl-1,3-benzothiazol-3-ium-2-yl)-N,N-dimethylaniline chloride (ThT)) is employed in this method. In other embodiments, the thioflavin compound/derivative is a compound other then Thioflavin T.

In particular embodiments, the invention provides a method of improving a measure of life span and/or health span, wherein the method entails administering to a subject an effective amount of one or more compounds selected from the group consisting of (2-(2-hydroxyphenyl)-benzoxazole (HBT), 2-(2-hydroxyphenyl)benzothiazole (HBX), 2-(2-aminophenyl)-1H-benzimidazole (BM), curcumin, and rifampicin and/or one or more derivatives thereof, whereby the measure of life span and/or health span is improved.

In an illustrative embodiment, the method entails co-administering Thioflavin T (4-(3,6-dimethyl-1,3-benzothiazol-3-ium-2-yl)-N,N-dimethylaniline chloride (ThT) and curcumin.

In various embodiments of the above-described methods, the improved measure of life span and/or health span can be one or more of the following: a reduction in frailty, an improvement in function in an age-related disability, the mitigation of a symptom of an age-related disease, and a delay in onset of frailty, age-related disability, or age-related disease, relative to the condition of the subject before administration of the compound or derivative or relative to a control population.

Examples of a reduction in frailty include: increased strength, weight gain, faster mobility, increased energy, increased levels of activity, increased endurance, and/or enhanced behavioral response to a sensory cue, wherein the reduction is relative to the condition of the subject before administration of the compound or derivative or relative to a control population. Other examples of a reduction in frailty include: a decrease in one or more inflammatory biomarkers, an improvement in glucose homeostasis, and a decrease in one of more biomarkers of clotting activation.

Examples of age-related disease include: osteoporosis, arthritis, cataracts, macular degeneration, and cardiovascular disease. Illustrative measures of mitigation of a symptom of an age-related disease include, but are not limited to, (an) improvement(s) in one or more of the following parameters: cholesterol level, triglyceride level, high density lipoprotein level, and blood pressure.

In certain embodiments, the improved measure of life span and/or health span can include a reduction in, a reversal of, or delay in onset of sarcopenia, relative to the condition of the subject before administration of the compound or derivative or relative to a control population.

In particular embodiments, the improved measure of life span and/or health span can include a reduction in, a reversal of, or delay in onset of an age-related increase in lipofuscin accumulation in one or more tissues selected from the group consisting of brain, heart, liver, spleen, and kidney, relative to the condition of the subject before administration of the compound or derivative or relative to a control population.

In certain embodiments, the methods described above are performed on a subject suffering from, or determined to be at risk for, one or more of the following: frailty, an age-related disability, or an age-related disease. For example, a subject may be determined to be suffering from, or determined to be at risk for, frailty. Such a determination can be made, e.g., by determining that the subject has at least three of the following symptoms: weakness, weight loss, slowed mobility, fatigue, low levels of activity, poor endurance, and impaired behavioral response to a sensory cue. A determination of frailty can also be made by determining that the subject has one or more of the following symptoms: an increase in one or more inflammatory biomarkers, glucose homeostasis impairment, and an increase in one of more biomarkers of clotting activation.

In particular embodiments, the methods described above are performed on a subject suffering from sarcopenia and/or lipofuscin accumulation in one or more of the following tissues: skeletal muscle, skin, brain, heart, liver, spleen, and kidney.

In illustrative embodiments, the improvement in a measure of life span and/or health span can include an enhanced ability to maintain homeostasis during the application of a stressor and/or a reduced time required to return to homeostasis after the application of a stressor. The stressor can be, e.g., drug-induced oxidative stress, exposure to heat, and/or exposure to cold. In variations of such embodiments, the subject is one who has been determined to have a reduced ability to maintain homeostasis during the application of a stressor and/or an extended time required to return to homeostasis after the application of a stressor, wherein the reduced ability or extended time is relative to the condition of the subject at a previous time or relative to a normal ability or time.

In certain embodiments, the measure of life span and/or health span can include the level and/or activity of a molecule that plays a role in protein trafficking, the autophagy pathway, ubiquitination, and/or lysozomal degradation of proteins. In variations of such embodiments, a method described above is performed on a subject who has been determined to have an abnormal level and/or activity of a molecule that plays a role in protein trafficking, the autophagy pathway, ubiquitination, and/or lysozomal degradation of proteins.

In particular embodiments, the measure of life span and/or health span can include the number of inclusion bodies in muscle tissue. In variations of such embodiments, a method described above is performed on a subject who has been determined to have abnormal inclusion bodies in muscle tissue.

In illustrative embodiments, the measure of life span and/or health span can include mitochondrial function and/or morphology. In variations of such embodiments, a method described above is performed on a subject who has been determined to have an abnormality in mitochondrial function and/or morphology.

In certain embodiments, the compound, or derivative thereof, is administered to a subject in more than one dose. An effective amount of compound/derivative can, e.g., range from about 0.001 μg/kg to about 10 μg/kg. The compound, or derivative thereof, can be administered, e.g., via any of the following routes of administration: intravenous, intraarterial, intrathecal, intradermal, intracavitary, oral, rectal, intramuscular, subcutaneous, intracisternal, intravaginal, intraperitonial, topical, buccal, and nasal.

In illustrative embodiments, the improvement in the measure of life span and/or health span can be at least about 40 percent, at least about 50%, at least about 60% relative to the condition of the subject before administration of the compound or derivative or relative to a control population.

A compound, or a derivative thereof, can, in certain embodiments, be co-administered with an effective amount of an additional agent that is useful for increasing a measure of life span and/or health span. Illustrative additional agents include an antioxidant, rapamycin, metformin, valproic acid, ethosuximide, trimethadione, 3,3-diethyl-2-pyrrolidinone, lithium, resveratrol, and derivatives thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-G. Thioflavin T (ThT) and functionally or structurally related compounds extend C. elegans lifespan. a) Dose-response Kaplan-Meier survival curves of synchronously ageing hermaphrodite wildtype (N2) populations exposed to 0 (control) to 500 μM ThT at 20° C. b) Percent gain in median survival of N2 populations cultured on 0-500 μM ThT and curcumin. c) In-linear plot of age-specific mortality rate with age for control and 50 μM ThT treated C. elegans. The Gompertz model was used to calculate the acceleration in age-specific mortality rate with age and initial mortality rate were calculated. d) Effect of 50 μM ThT and 100 μM curcumin on locomotory ability of N2 worms evaluated as the average number of body bends/20 s in 15 individual worms throughout life (upper panel) and after twelve days of treatment with ThT and curcumin. Data are presented as bends/min and represent the average of three independent experiments. *p<0.0001. e) Dose-response Kaplan-Meier survival curves of synchronously ageing hermaphrodite N2 populations exposed to 0 (control) to 1 μM of BM, f) HBX and g) HBT at 20° C. (−) Control, (▪) 1 nM, () 10 μM, (▴) 100 μM, (▾) 1 μM. Graphs are representative of three independent experiments.

FIG. 2. Thioflavin-T (ThT) extends C. elegans lifespan in absence of FUdR. Kaplan-Meier survival curves of synchronously ageing hermaphrodite wildtype (N2) populations exposed to 0 (control) and 50 μM ThT at 20° C. Graph is representative of 4 independent experiments. Average increase in median lifespan was 40% ranging from 30-70%.

FIG. 3A-C. Chemical structures of some protein aggregate-binding dyes. a) Thioflavin T, b) Curcumin, c) Rifampicin.

FIG. 4A-B. Effect of Curcumin and Rifampicin on C. elegans lifespan. Dose-response Kaplan-Meier survival curves of synchronously ageing hermaphrodite wildtype (N2) populations exposed to 0 (control) through 500 μM of a) Curcumin and b) Rifampicin at 20° C. (−) Control, () 1 μM, (▴) 10 μM, (▾) 50 μM, (♦) 100 μM, (X) 500 μM. Graphs are representative of two independent experiments.

FIG. 5A-B. ThT and curcumin effects are non-additive on C. elegans lifespan. a) Effect of 50 μM ThT with the addition of 0 (blue), 25 (orange), 50 (green) and 100 (yellow) μM curcumin on lifespan of synchronous populations of N2 worms at 20° C. b) Fraction gain in median survival of N2 populations cultured on 50 μM ThT plus 25, 50 and 100 μM curcumin. Bars represent the mean±the SEM of four independent experiments.

FIG. 6A-C. Chemical structures of ThT-like compounds. a) 2-(2-aminophenyl)-1H-benzimidazole (BM), b) 2-(2-hydroxyphenyl)benzoxazole (HBT) and c). 2-(2-hydroxyphenyl)benzothiazole (HBX). These compounds also differ in their physicochemical properties, particularly polarity and partition coefficient. These differences are likely to account for variations in the pharmacokinetic behavior (e.g., the traffic across biological membranes) of HBT, HBX and BM as compared with ThT and consequently the effective dose.

FIG. 7A—F. ThT and curcumin rescue paralysis phenotypes and prevent protein aggregation in vivo. Protection of the paralysis phenotype elicited by 25 μM ThT, 50 μM ThT, 100 μM curcumin in a) CL4176 (*p<0.001, **p<0.0001) and b) AM140 (*p<0.05, **p<0.01) after 1 and 8 days at 25° C. respectively. Bars represent the mean±SEM of four independent experiments per duplicate. c) Temperature-sensitive strain HE250 after 36 h at 25° C. showing the typical paralysis phenotype (left upper panel) and the rescue elicited by 50 μM ThT (right upper panel). Arrows show the halos of clearance in the bacterial lawn characteristic of paralyzed worms. Protection (±SD) of the HE250 paralysis phenotype by 50 μM ThT, 100 μM curcumin and 100 μM rifampicin (lower panel). *p<0.0001, **p<0.001. n=4 independent experiments d) Perlecan immunolocalization showing disruption/aggregation pattern after 24 h at 25° C. and the suppression of disruption by 50 μM ThT treatment. 16 of 20 worms exhibited similar perlecan distribution in 3 independent experiments. Arrows indicate perlecan aggregates. Bar=30 μm. e) Immunolocalization of aggregation-prone soluble oligomeric protein (A11 antibody, red) and Aβ₃₋₄₂ (green) in presence or absence of 50 μM ThT in strain CL4176. Bars represent the mean±SEM, 11 worms per group, in three independent experiments. *p<0.0001.f) Immunolocalization of aggregation-prone soluble oligomeric protein (A 11 antibody) in presence or absence of 50 μM ThT on 11 days old wildtype N2 worms. Bars represent the mean±SEM, 11 worms per group, of three independent experiments. *p<0.0001. Bar=20 μm.

FIG. 8A-B. ThT rescues paralysis phenotype in an Aβ worm model of protein aggregation even when added after the aggregation induction. Prevention of paralysis phenotype elicited by 50 μM ThT in worms continuously exposed and after 18 h at the restrictive temperature (25° C.) in a) CL4176 dvIs27[myo-3::Aβ(1 to 42)-let 3′UTR(pAF29); pRF4 (rol-6(su1006))] (*p<0.0001) and b) HE250 [unc-52(e669su250)II] (*p<0.05, **p<0.001) scored after 36 h at 25° C. Error bars represent the mean±SEM of four independent experiments.

FIG. 9A-B. ThT protects against sarcomere structural disruption elicited by UNC-54 temperature-sensitive structural muscle protein aggregation. a) Paramyosin immunolocalization showing a typical disruption/aggregation pattern after 24 h at 25° C. (left panel) and the prevention of this aggregation elicited by 50 μM ThT treatment (right panel) in (unc-54(e1157)I). Arrows show sarcomere disruption and paramyosin aggregation, arrowheads show intact muscle sarcomeres. Bar=20 μm. b) RT-PCR in single worm to detect the RNA levels of unc-52 and unc-54 after 3, 6 and 12 days of ThT treatment. ThT treatment increases unc-54 RNA levels after 3, but not at 6 or 12 days of treatment (left panel) and decreases the levels of unc-52 at 12 days of treatment (right panel) (*p<0.01, **p<0.001).

FIG. 10A-B. ThT prevents the loss of function of folding sensors expressed in different tissues with temperature-sensitive (ts) phenotypes. Temperature sensitive strains that harbor missense mutations in different tissues were assayed for a) ethanol sensitivity (CW152 gas-1(fc21) X, gas-1) and b) levamisole resistance (ZZ26 unc-63 (x26)I, unc-63). ThT decreases the ethanol sensitivity and increases levimisole resistance associated to misfolding/aggregation of gas-1 and unc-64 mutants, respectively, after 3 days of treatment. Graphs are representative of three independent experiments.

FIG. 11A-B. ThT produces small changes in pharyngeal pumping in N2 worms and increases lifespan on a dietary restriction model. a) Pharyngeal pumping rates of wildtype animals raised on 50 μM ThT measured at 3 (left panel) and 6 days of treatment (right panel). Pumping rates of 15 individuals were scored and the averages of three independent experiments are shown (*p<0.01). b) 1 and 10 μM ThT increase lifespan of N2 worms raised on 1×10⁹ cfu/ml at 20° C. as compared to untreated worms (left panel). Percent gain in median survival of N2 populations cultured on 0-50 μM ThT (right panel). Bars are representative of 2 to 4 independent experiments per duplicate.

FIG. 12. ThT is localized in tissues with A11 positive puncta and Aβ peptide material. a) A11 (red) and Aβ (green) detected by immunolocalization in CL4176 worms treated with 50 μM ThT (blue) were analyzed under two-photon excitation microscopy after 36 h at 25° C. ThT was detected in tissues with A11 positive puncta (magenta indicates overlapping signals) and with some Aβ peptide cytosolic oligomers (light blue). b) Magnification of the inset shown in a). Bar=10 μm.

FIG. 13. Dependency of proteostasis factors on ThT suppression of protein-aggregation-associated paralysis in C. elegans. The expression of genes encoding proteostatic factors were knocked down by RNAi feeding in HE250 [unc-52(e669su250)II] in the presence or absence of 50 μM ThT and the paralysis phenotype was scored after 36 h. Where no difference was observed, it is possible the RNAi treatment was not optimal. Proportion of worms paralyzed is plotted (mean±SEM).*p<0.01, ***p<0.0001 vs CV (control vector).

FIG. 14A-E. ThT and curcumin enhancement of lifespan depends on the heat shock factor-1 (HSF-1) transcription factor but not on DAF-16. Effect of 50 μM ThT and 100 μM curcumin on a) PS3551 [hsf-1(sy441)I], b) CF1038 [daf-16(mu86)I] and c) DA465 [eat-2(ad465)II]. Graphs are representative of three independent experiments. d) ThT and curcumin treatment does not produce DAF-16::GFP relocalization as compared to control strain z1s356 IV [daf-16::daf-16-gfp+rol-6] (upper left). Control strain under heat-shock produces a clear relocalization of DAF-16::GFP (upper right). e) ThT treatment (lower panel) does not induce SKN-1::GFP relocalization as compared to control strain (upper panel).

FIG. 15. Heat shock factor-1 (HSF-1) transcription factor dependency of ThT lifespan enhancement assayed by RNAi knockdown of hsf-1 on PS3551. Lifespan increase elicited by 50 μM ThT treatment is abolished in PS3551 (sy441) background and no changes were found by an additional hsf-1 knockdown on this background. Graphs are representative of three independent experiments.

FIG. 16A-B. ThT treatment induces the expression of some HSPs but does not changes the levels of HSF-1. a) ThT (50 μM) and curcumin (100 μM) increase the HSP-16.2 levels and ThT slightly increases HSP-70 levels after 4 and 7 days of treatment (left panel). ThT Treatment also increases the RNA levels of two members of the HSP-70 family, hsp-6 and chn-1, after 3 and 6 days of treatment (right panel). b) ThT treatment increases HSF-1 levels (left panel) but no changes were detected in the RNA levels after 3, 6 and 12 days of treatment (right panel). Western blots are representative of at least three independent experiments. (*p<0.05, **p<0.01, ***p<0.01).

FIG. 17. Effect of ThT on the lifespan of age-1 (hx546) mutant worms. Effect of 50 μM ThT (▪) on lifespan of synchronous populations of age-1 (hx546)II () worms at 25° C. Black line corresponds to the lifespan of age-matched N2 wildtype controls. Graph is representative of two independent experiments per duplicate.

FIG. 18. Heat shock results in nuclear localization of DAF-16::GFP after ThT or curcumin treatment. daf-16::daf-16-gfp worms treated with 25 μM ThT, 50 μM ThT and 100 μM curcumin at 20° C. were heat shocked at 35° C. and DAF-16::GFP nuclear localization was observed in all cases.

### DEFINITIONS

Terms used in the claims and specification are defined as set forth below unless otherwise specified.

The phrases “an effective amount” and “an amount sufficient to” refer to amounts of a biologically active agent that produce an intended biological activity.

The term “thioflavin compound” is used herein to refer to any compound that has one of structures A-E:

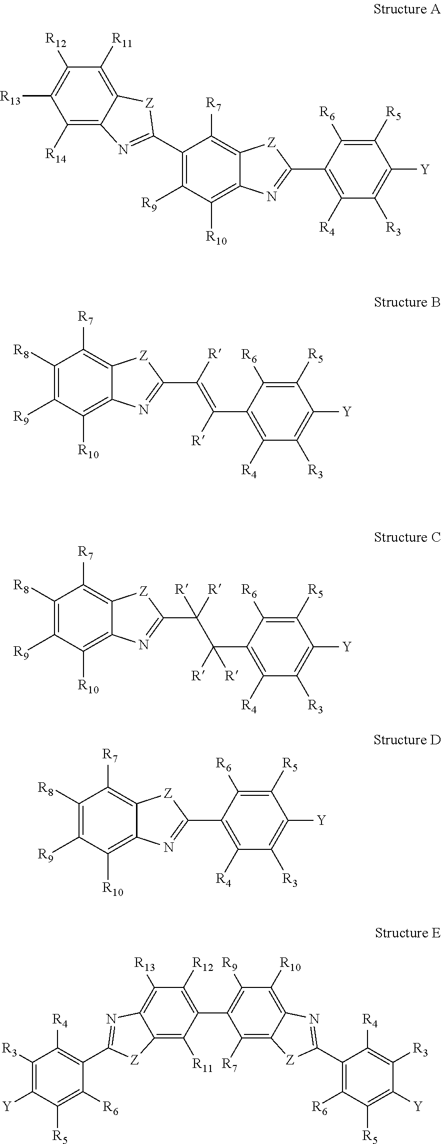

wherein Z is S, NR′, 0 or CR′ in which case the correct tautomeric form of the heterocyclic ring becomes an indole in which R′ is H or a lower alkyl group:

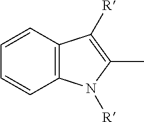

wherein Y is NR′R², OR², or SR²;
wherein the nitrogen of

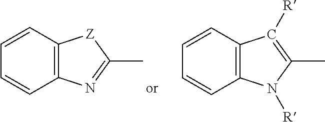

is not a quartemary amine;
or an thioflavin compound having one of structures F-J or a water soluble, non-toxic salt thereof:

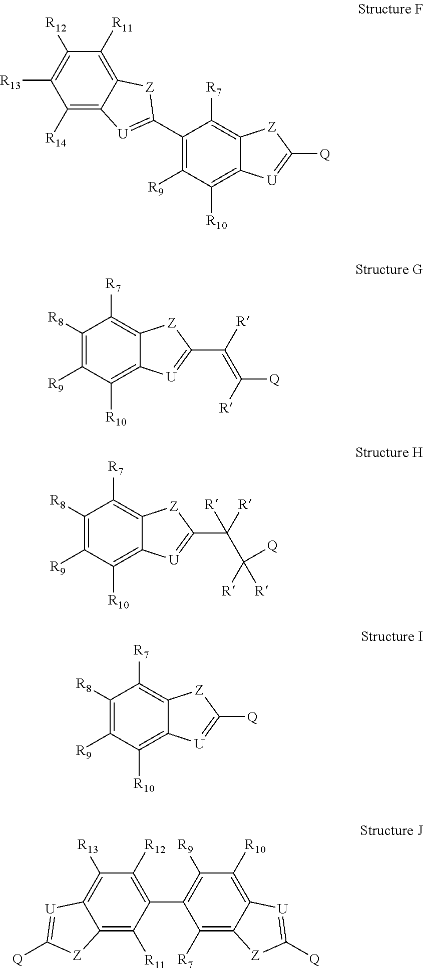

wherein each Q is independently selected from one of the following structures:

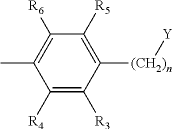

wherein n=0, 1, 2, 3 or 4,

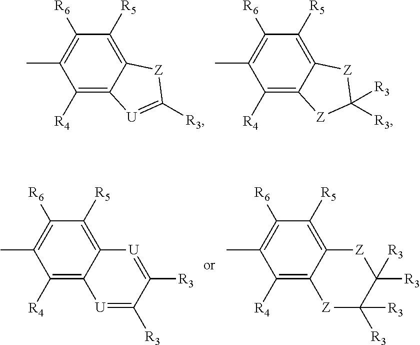

wherein Z is S, NR′, O, or C(R′)₂ in which R′ is H or a lower alkyl group;
wherein U is CR′ (in which R′ is H or a lower alkyl group) or N (except when U═N, then Q is not

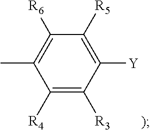

wherein Y is NR¹R², OR², or SR²;
wherein the nitrogen of

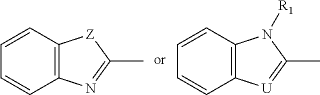

is not a quaternary amine;
wherein each R¹ and R² independently is selected from H, a lower alkyl group, (CH₂), OR′ (wherein n=1, 2, or 3), CF₃, CH₂—CH₂X, CH₂—CH₂—CH₂X (wherein X═F, Cl, Br or I), (C═O)—R′, Rₚₕ, and (CH₂), Rₚₕ (wherein n=1, 2, 3, or 4 and Rₚₕ represents an unsubstituted or substituted phenyl group with the phenyl substituents being chosen from any of the non-phenyl substituents defined below for R³-R¹⁴ and R′ is H or a lower alkyl group);
and wherein each R³-R¹⁴ independently is selected from the group consisting of H, F, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, N0₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from any of the non-phenyl substituents defined for R¹-R¹⁴ and wherein R′ is H or a lower alkyl group), a tri-alkyl tin and a chelating group (with or without a chelated metal group) of the form W-L or V—W-L, wherein V is selected from: —COO—, —CO—, —CH₂O— and —CH₂NH—; W is —(CH₂)ₙ where n=0, 1, 2, 3, 4, or 5; and L is:

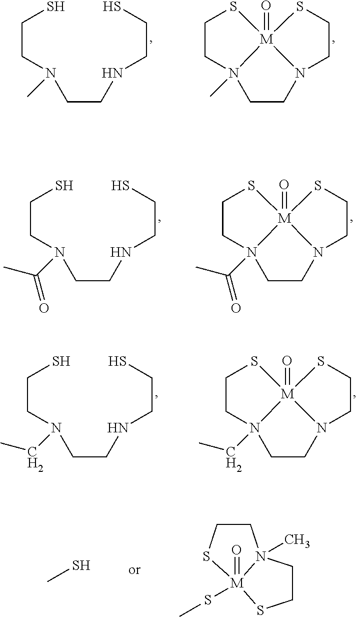

wherein M is selected from Tc, Re, Zn, Cu, Ni, V, Mn, Fe, Cr and Ru;
or wherein each R¹ and R² is a chelating group (with or without a chelated metal group) of the form W-L, wherein W is —(CH₂)ₙ where n=2, 3, 4, or 5; and L is:

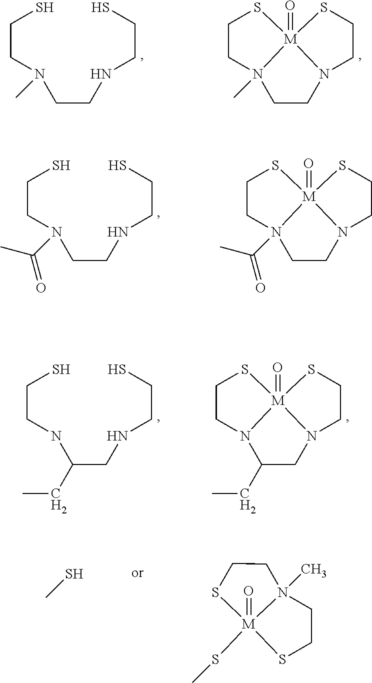

wherein M is selected from Tc and Re;
or wherein each R¹-R¹⁴ independently is selected from a chelating group (with or without a chelated metal ion) of the form W-L and V—W-L, wherein V is selected from —COO—, and —CO—; W is —(CH₂)ₙ where n=0, 1, 2, 3, 4, or 5; L is:

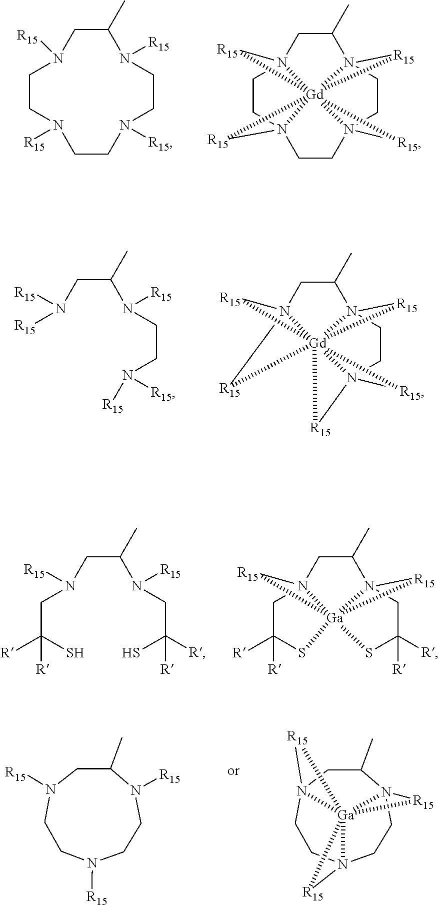

and wherein R15 independently is selected from the following:

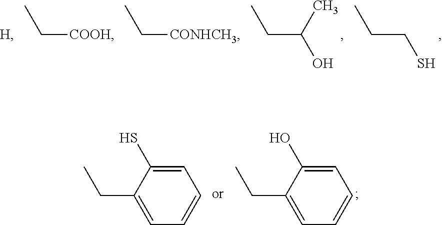

or a chelating compound (with or without a chelated metal group) or a water soluble, non-toxic salt thereof of the form:

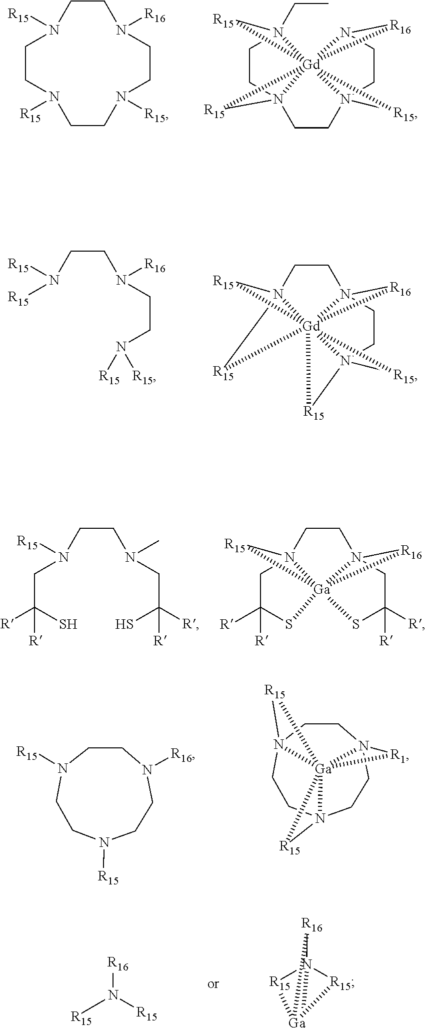

wherein R¹⁵ independently is selected from the following:

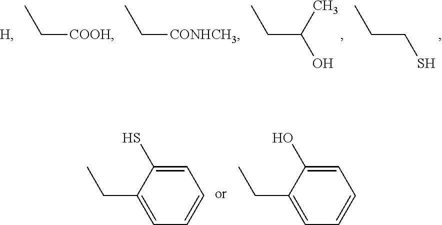

### and R¹⁶ is

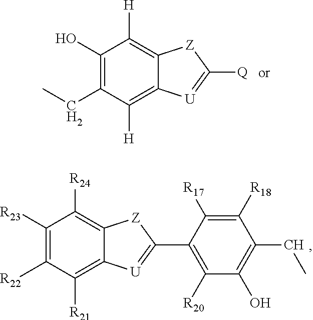

wherein
Q is independently selected from one of the following structures:

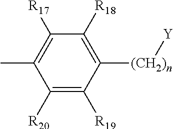

wherein n=0.1, 2, 3 or 4,

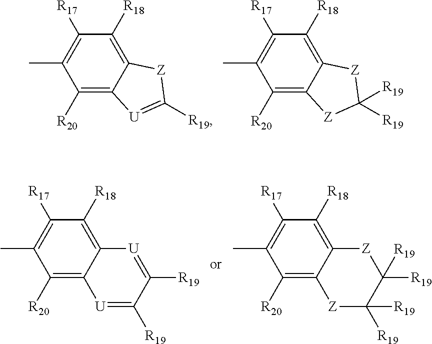

wherein Z is S, NR′, O, or C(R′)₂ in which R′ is H or a lower alkyl group;
wherein U is N or CR′;
wherein Y is NR¹R², OR², or SR²;
wherein each R¹⁷-R²⁴ independently is selected from H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ and CR₂′—CR₂′—Rₚₕ (wherein Rₚₕ represents an unsubstituted or substituted phenyl group with the phenyl substituents being chosen from any of the non-phenyl substituents defined for R¹⁷-R²⁰ and wherein R′ is H or a lower alkyl group).

The term “derivative” used with reference to a compound encompasses any salt, ester, amide, prodrug, or other derivative of the compound, that has at least one pharmacological effect of the compound that renders it useful in one or more of the methods of the invention, and is pharmaceutically acceptable.

The term “health span” refers to the period of time during which an individual meets one or more selected measures of health span. An increase in “health span” refers to an extension in the period of health, according to such measures, as compared to the period of health in a control population. An increase in health span can be measured, e.g., by determining the length of time that an individual continues to meet the selected measure(s) of health span. Alternatively, an increase in health span can be determined by measuring a degree of improvement in one or more selected measures of health span that is correlated with and increase in the length of time that and individual continues to meet the selected measures of health span.

The term “frailty” refers to a condition that can be characterized by (typically, three or more) symptoms selected from weakness, weight loss, slowed mobility, fatigue, low levels of activity, poor endurance; and impaired behavioral response to a sensory cue. Frailty can also be characterized by an increase in one or more inflammatory biomarkers, glucose homeostasis impairment, and/or an increase in one of more biomarkers of clotting activation. Another hallmark of frailty is “sarcopenia,” which refers to age-related loss of muscle mass.” Frailty can also refer to a reduced ability to maintain homeostasis during the application of a stressor and/or an increase in the time required to return to homeostasis after the application of a stressor. Frailty can also include a decline in mitochondrial function, typically with changes in respiration, and/or morphological aberrations in mitochondria.

An “age-related disability,” refers to any physical or mental incapacity associated with normal aging, such as, for example, an age-related decline in near vision.

An “age-related disease” refers an abnormal condition characterized by a disordered or incorrectly functioning organ, part, structure, or system of the body that occurs more frequently in the aged.

As used herein, a “control population,” refers to a population that has not been treated with a thioflavin compound or derivative, wherein the members of that population have one or more characteristics and/or conditions of a subject being treated with a thioflavin compound or derivative. Thus, for example, if a subject is being treated for frailty, the relevant control population would have frailty; and if a subject is being treated for and age-related disability or disease, the relevant control population would have the same disability or disease.

The term “inflammatory biomarker” refers to an endogenous condition, often the presence, level, and/or form of a molecule, that indicates the presence of inflammation. For, example, C-reactive protein (CRP), is an inflammatory biomarker that has been shown to predict future cardiovascular events in individuals with and without established cardiovascular disease (CVD). Biomarkers implicated in the inflammatory process leading to atherothrombosis, include, for example, CRP, adiponectin, monocyte chemoattractant protein 1 (MCP-1), CD40 ligand and lipoprotein-associated phospholipase A(2) (Lp-PLA(2)).

The term “glucose homoestasis” refers to the state of, or tendency toward, normal (non-pathological) glucose levels, which vary appropriately in response to various stimuli. Illustrative measure of glucose homeostasis include meal-stimulated insulin, glucose, and glucagon-like peptide-1 (GLP-1) levels.

The term “biomarker of clotting activation” refers to an endogenous condition, often the presence, level, and/or form of a molecule, that indicates activation of the pathway leading to the formation of a blood clot. Illustrative biomarkers of clotting activation include, for example, prothrombin fragments 1 and 2 (F1+2), thrombin-antithrombin complex (TAT), and fibrin degradation products (D-dimer).

The term “lipofuscin” refers to lipopigments that are made up of fats and proteins. Lipofuscin take on a greenish-yellow color when viewed under an ultraviolet light microscope. Lipofuscins can build up in neuronal cells and many organs, including the brain, liver, spleen, myocardium, and kidneys, excessive accumulation can lead to neurodegenerative disorders, such as neuronal ceroid lipofuscinoses.

The term “autophagy pathway” refers to a self-cannibalisation pathway that is one of the main mechanisms for maintaining cellular homeostasis. Mediated via the lysosomal degradation pathway, autophagy is responsible for degrading cellular proteins and cellular organelles, recycling them to ensure cell survival. Autophagy includes three processes: microautophagy, macroautophagy and chaperone-mediated autophagy. “Microautophagy” is the transfer of cytosolic components into the lysosome by direct invagination of the lysosomal membrane and subsequent budding of vesicles into the lysosomal lumen. “Macroautophagy” involves formation of a double-membrane structure called the autophagosome which sequesters cytosolic material and delivers it to the lysosome for degradation. This degradation can be selective (i.e., specifically removing damaged mitochondria, while sparing normal functioning ones); however, degradation of soluble cytosolic proteins is non-selective. “Chaperone-mediated autophagy” (CMA) is characterized by its selectivity in degrading specific substrates (cytosolic proteins). Genetic screens in yeast (S. cerevisiae) have led to the identification of over ˜30 autophagy-related genes (ATG-genes), many of which have identified mammalian homologues. Examples of the latter include hAPG5, Beclin-1, HsGSA7/haPG7, MAP1LC3, hAPG12, PTEN, and LAMP-2.

The term “ubinquitination” refers to the tagging of proteins for selective destruction in proteolytic complexes called proteasomes by covalent attachment of ubiquitin, a small, highly conserved protein. An isopeptide bond links the terminal carboxyl of ubiquitin to the ε-amino group of a lysine residue of a “condemned” protein. Three enzymes are involved. Initially, the terminal carboxyl group of ubiquitin is joined in a thioester bond to a cysteine residue on ubiquitin-activating enzyme (E1). This step is dependent on ATP. The ubiquitin is then transferred to a sulfhydryl group on a ubiquitin-conjugating enzyme (E2). A ubiquitin-protein ligase (E3) the transfers ubiquitin from E2 to the ε-amino group of a lysine residue of a protein recognized by that E3, forming an isopeptide bond. More ubiquitins may be added to form a chain of ubiquitins. The terminal carboxyl of each ubiquitin is linked to the ε-amino group of a lysine residue (Lys29 or Lys48) of the adjacent ubiquitin in the chain. A chain of four or more ubiquitins targets proteins for degradation in proteasomes.

As used herein, “inclusion bodies” refer to nuclear or cytoplasmic aggregates of stainable substances, typically proteins. Proteins in inclusion bodies may be misfolded. “Inclusion body myocitis” refers to an age-related, inflammatory muscle disease, characterized by slowly progressive weakness and wasting of both distal and proximal muscles, most apparent in the muscles of the arms and legs. In sporadic inclusion body myositis, two processes, one autoimmune and the other degenerative, appear to occur in the muscle cells in parallel. The inflammation aspect is characterized by the cloning of T cells that appear to be driven by specific antigens to invade muscle fibers. The degenerative aspect is characterized by the appearance of vacuoles and deposits of abnormal proteins in muscle cells and filamentous inclusions.

The term “protein trafficking” refers to the movement of proteins within a cell. For example, nascent proteins may be targeted to the cytosol, mitochondria, peroxisomes or chloroplasts. These proteins (if encoded in the nucleus) are synthesized on free ribosomes. However, proteins destined for secretion, for the lumen of the endoplasmic reticulum (ER), Golgi or lysosomes, or for the membrane of any of these organelles or the plasma membrane, are synthesized on the membrane-bound ribosomes of the rough ER. They are then targeted to the appropriate cellular compartment. It is estimated that over 100 inherited human diseases, such as cystic fibrosis, lysosomal storage diseases, and long QT syndrome, are due to protein trafficking defects, typically caused by mutations in secreted proteins which prevent proper folding of the protein. These mutant proteins fold inefficiently and, thus, fail to exit the ER. This produces a “loss of function” phenotype. The misfolded proteins are detected by a quality control system in the ER and are degraded by the ubiquitin proteasome system.

The term “co-administer,” when used in reference to the administration of thioflavin compounds and other agents, indicates that the two agents are administered so that there is at least some chronological overlap in their physiological activity on the organism. Thus, the thioflavin compound can be administered simultaneously and/or sequentially with the other agent. In sequential administration, there may even be some substantial delay (e.g., minutes or even hours or days) before administration of the second agent as long as the first administered agent is exerting some physiological effect on the organism when the second administered agent is administered or becomes active in the organism.

The term “therapy” or “treatment” as used herein, encompasses the treatment of an existing condition as well as preventative treatment (i.e., prophylaxis). Accordingly, “therapeutic” effects and applications include prophylactic effects and applications, respectively.

I. Method of Improving a Measure of Life Span and/or Health Span

A. In General

In certain embodiments, the invention provides a method of improving a measure of life span and/or health span, wherein the method entails administering an effective amount of a thioflavin compound, or derivative thereof, to a subject.

In illustrative embodiments, the invention provides a method of improving a measure of life span and/or health span, wherein the method entails administering to a subject an effective amount of one or more of the following compounds: (2-(2-hydroxyphenyl)-benzoxazole (HBT), 2-(2-hydroxyphenyl)benzothiazole (HBX), 2-(2-aminophenyl)-1H-benzimidazole (BM), curcumin, and rifampicin and/or one or more derivatives thereof. Without being limited to a particular mechanism, it is believed that these compounds are amyloid-binding compounds that slow aging by modulating protein homeostasis.

Because the compounds and derivatives described herein are used therapeutically, rather than diagnostically, according to the methods of the invention, the compounds or derivatives are typically administered in more than one dose. In particular embodiments, the administration of one or more compounds or derivatives improves the measure of life span and/or health span (“life/health span”) at least about 5, 10 15, 20 25, 30 35, 40 45, 50, 55, 60, 65, 70, 75, 80, 85, 90, 95 percent or more, relative to the condition of the subject before treatment or relative to a control population.

B. Thioflavin Compounds

Thioflavin compounds useful in the method have one of structures A-E:

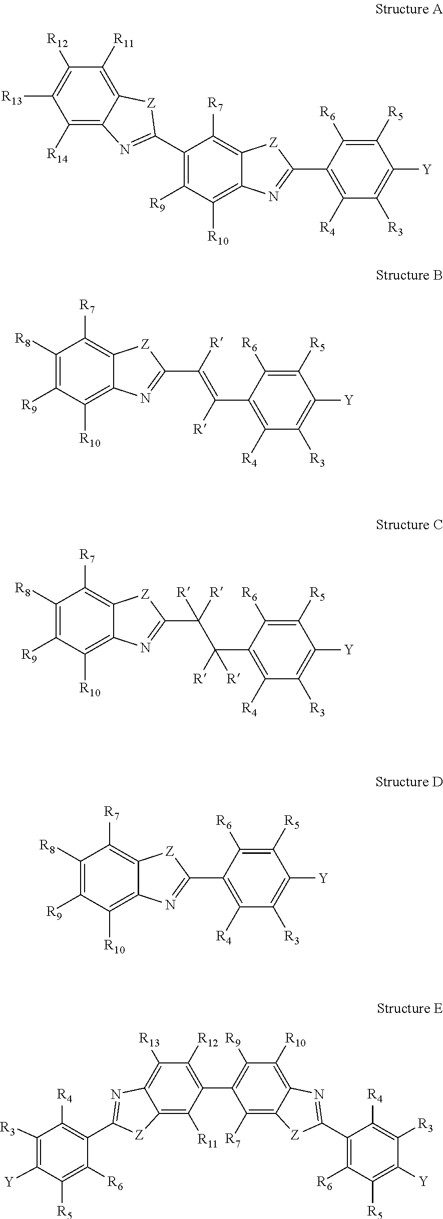

wherein Z is S, NR′, 0 or CR′ in which case the correct tautomeric form of the heterocyclic ring becomes an indole in which R′ is H or a lower alkyl group:

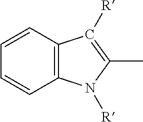

wherein Y is NR¹R², OR², or SR²;
wherein the nitrogen of

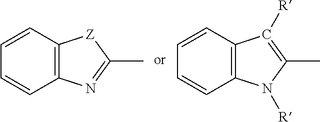

is not a quarternary amine;
or a thioflavin compound having one of structures F-J or a water soluble, non-toxic salt thereof:

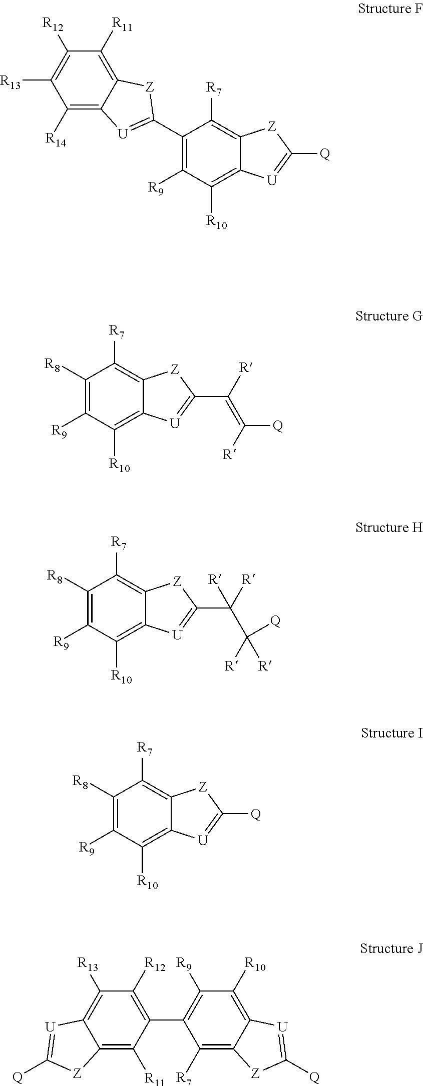

wherein each Q is independently selected from one of the following structures:

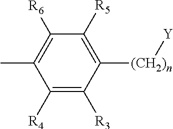

wherein n=0, 1, 2, 3 or 4,

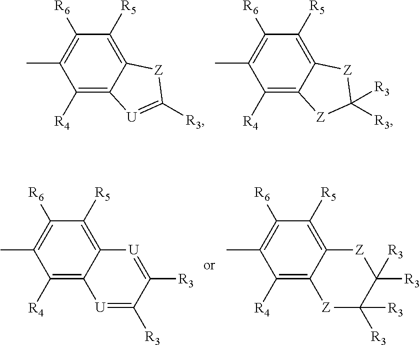

wherein Z is S, NR′, 0, or C(R′)₂ in which R′ is H or a lower alkyl group;
wherein U is CR′ (in which R′ is H or a lower alkyl group) or N (except when U═N, then Q is not

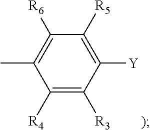

wherein Y is NR¹R², OR², or SR²;
wherein the nitrogen of

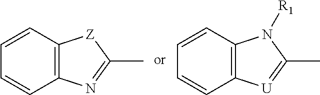

is not a quaternary amine;
wherein each Wand R² independently is selected from H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from any of the non-phenyl substituents defined below for R³-R¹⁴ and R′ is H or a lower alkyl group);
and wherein each R³-R¹⁴ independently is selected from H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, N0₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from any of the non-phenyl substituents defined for R¹-R¹⁴ and wherein R′ is H or a lower alkyl group), a tri-alkyl tin and a chelating group (with or without a chelated metal group) of the form W-L or V—W-L, wherein V is selected from: —COO—, —CO—, —CH₂O— and —CH₂NH—; W is —(CH₂)ₙ where n=0, 1, 2, 3, 4, or 5; and L is:

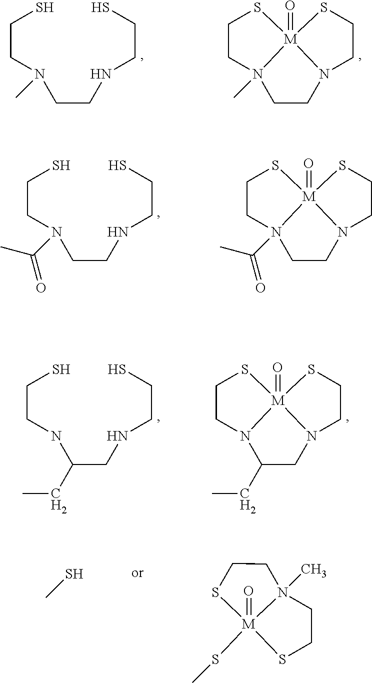

wherein M is selected from Tc, Re, Zn, Cu, Ni, V, Mn, Fe, Cr and Ru;
or wherein each R′ and R² is a chelating group (with or without a chelated metal group) of the form W-L, wherein W is —(CH₂)ₙ where n=2, 3, 4, or 5; and L is:

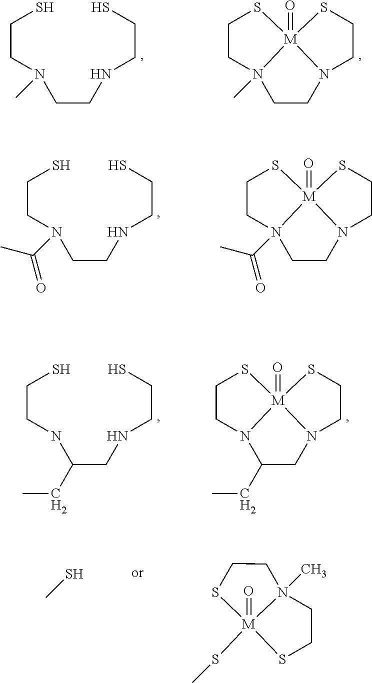

wherein M is selected from Tc and Re;
or wherein each R¹-R¹⁴ independently is selected from a chelating group (with or without a chelated metal ion) of the form W-L and V—W-L, wherein V is selected from —COO—, and —CO—; W is —(CH₂)ₙ where n=0, 1, 2, 3, 4, or 5; L is:

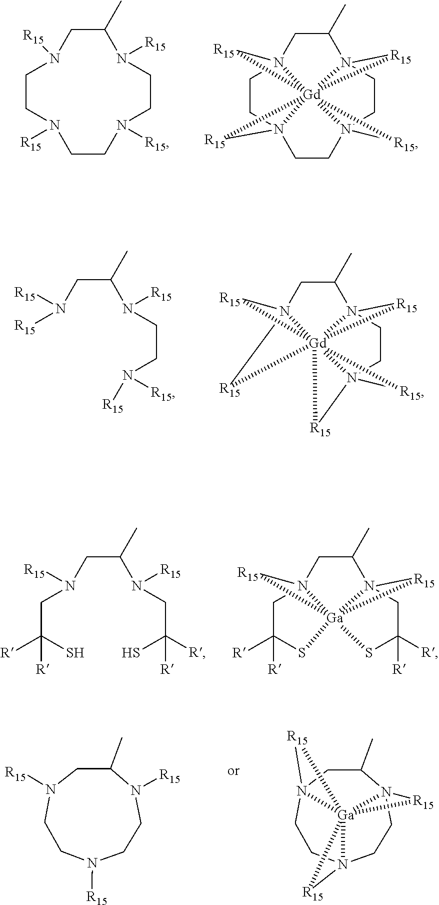

and wherein R¹⁵ independently is selected from the following:

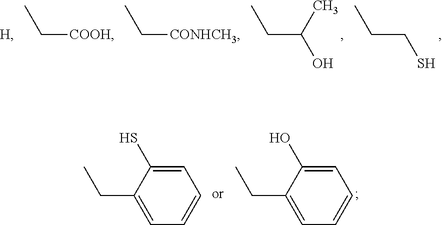

or a chelating compound (with or without a chelated metal group) or a water soluble, non-toxic salt thereof of the form:

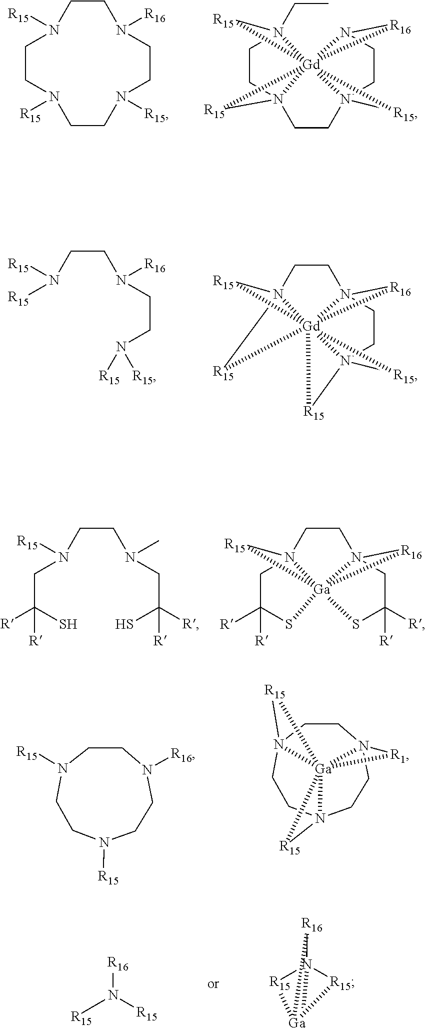

wherein R¹⁵ independently is selected from the following:

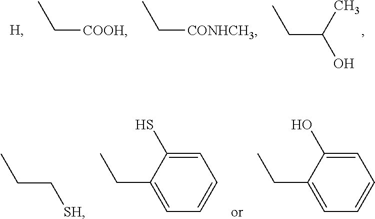

### and R¹⁶ is

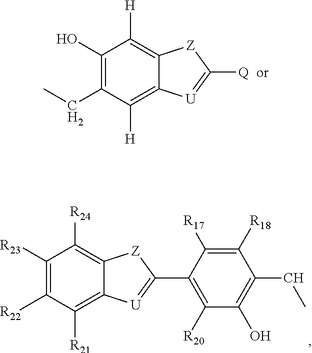

wherein
Q is independently selected from one of the following structures:

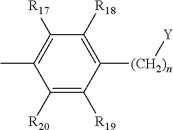

wherein n=0, 1, 2, 3 or 4,

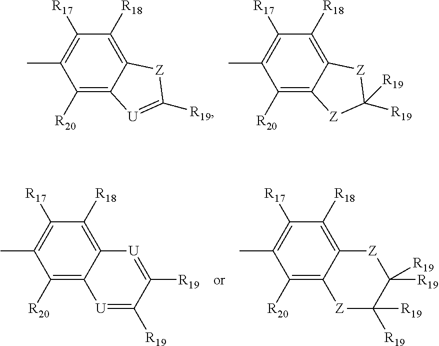

wherein Z is S, NR′, 0, or C(R′)₂ in which R′ is H or a lower alkyl group;
wherein U is N or CR′;
wherein Y is NR¹R², OR², or SR²;
wherein each R¹⁷-R²⁴ independently is selected from H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ and CR₂′—CR₂′—Rₚₕ (wherein Rₚₕ represents an unsubstituted or substituted phenyl group with the phenyl substituents being chosen from any of the non-phenyl substituents defined for R¹⁷-R²⁰ and wherein R′ is H or a lower alkyl group).

In an illustrative embodiment, Thioflavin T (4-(3,6-dimethyl-1,3-benzothiazol-3-ium-2-yl)-N,N-dimethylaniline chloride (ThT)) can be employed in the methods described herein.

The invention also encompasses the use of any member of the above-described genus of thioflavin compounds, provided that the genus excludes Thioflavin T (4-(3,6-dimethyl-1,3-benzothiazol-3-ium-2-yl)-N,N-dimethylaniline chloride (ThT).

In certain embodiments, Z═S, Y═N, R¹═H; and

wherein when the thioflavin compound is structure A or E, then R² is selected from a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rph, and (CH₂)nRₚₕ wherein n=1, 2, 3, or 4;

wherein when the thioflavin compound is structure B, then R² is selected from (CH₂)ₙOR′ (wherein n=1, 2, or 3, and where when R′═H or CH₃, n is not 1), CF₃, CH₂—CH₂X and CH₂—CH₂—CH₂X (wherein X═F, Cl, Br or I);

wherein when the thioflavin compound is structure C, then R² is selected from a lower alkyl group, (CH₂)ₙOR′ (wherein n=1, 2, or 3, CF₃), CH₂—CH₂X, CH₂—CH₂—CH₂X (wherein X═F, Cl, Br or I), (C═O)—H, Rₚₕ, and (CH₂)ₙRₚₕ wherein n=1, 2, 3, or 4; and

wherein when the thioflavin compound is structure D, then R² is selected from (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4) wherein when R₂ is CH₂Rₚₕ R8 is not CH₃.

In variations of these certain embodiments, at least one of the substituents R³-R¹⁴ is selected from ON, OCH₃, OH and NH₂.

In various embodiments of structures A-E, Z═S, Y═N, R′═H, R¹═H, R²═CH₃ and R³-R¹⁴ are H.

In various embodiments of structures A-E, Z═S, Y=0, R′═H, R²═CH₃ and R³-R¹⁴ are H.

In various embodiments of structures A-E, Z═S, Y═N, R′═H, R¹⁻⁴═H, R⁵═I, and R⁶-R¹⁴ are H.

In various embodiments of structures A-E, Z═S, Y═N, R′═H, R¹⁻⁴═H, R⁵═I, R⁸═OH and R⁶-R⁷ and R⁹-R¹⁴ are H.

In various embodiments of structures A-E, Z═S, Y═N, R′═H, R¹═H, R²═CH₂—CH₂—CH₂—F and R³-R¹⁴ are H.

In various embodiments of structures A-E, Z═S, Y═O, R′═H, R²═CH₂—CH₂—F and R³-R¹⁴ are H.

In various embodiments of structures A-E, Z═S, Y═N, R′═H, R¹⁻⁷═H, R⁸═O—CH₂—CH₂—F and R⁹-R¹⁴ are H.

In various embodiments of structures A-E, Z═S, Y═N, R′═H, ═CH₃, R²⁻⁷═H, R⁸═O—CH₂—CH₂—F and R⁹— R¹⁴ are H.

In various embodiments of structures F-J, Z═S, Y═N, R′═H, R′═H, R²═CH₃ and R³-R¹⁴ are H.

In various embodiments of structures F-J, Z═S, Y═O, R′═H, R²═CH₃ and R³-R¹⁴ are H.

In various embodiments of structures F-J, Z═S, Y═N, R′═H, R¹⁻⁴═H, R⁵═I, and R⁶-R¹⁴ are H.

In various embodiments of structures F-J, Z═S, Y═N, R′═H, R¹⁻⁴═H, R⁵═I, R⁸═OH and R⁶-R⁷ and R⁹-R¹⁴ are H.

In various embodiments of structures F-J, Z═S, Y═N, R′═H, R¹═H, R²═CH₂—CH₂—CH₂—F and R³-R¹⁴ are H.

In various embodiments of structures F-J, Z═S, Y═O, R′═H, R²═CH₂—CH₂—F and R³-R¹⁴ are H.

In various embodiments of structures F-J, Z═S, Y═N, R′═H, R¹⁻⁷═H, R⁸═O—CH₂—CH₂—F and R⁹-R¹⁴ are H.

In various embodiments of structures F-J, Z═S, Y═N, R′═H, R¹═CH₃, R²⁻⁷═H, R⁸═O—CH₂—CH₂—F and R⁹-R¹⁴ are H.

In embodiments wherein the thioflavin compound is selected from structure B, structure C and structure D, R¹═H, R²═CH₃ and R⁸ is selected from CN, CH₃, OH, OCH₃ and NH₂. In variations of these embodiments, R³-R⁷ and R⁹-R¹⁴ are H.

The thioflavin compounds described above can be produced as described in PCT Publication No. WO 02/16333, which is hereby incorporated by reference for its description of thioflavin compounds and their production.

C. Measures of Life Span and/or Health Span

Any suitable measure of life/health span can be employed in the methods described herein. In certain embodiments, an improvement in life span and/or health span can be detected as a reduction in frailty, an improvement in function in an age-related disability, the mitigation of a symptom of an age-related disease, and/or a delay in onset of frailty, age-related disability, or age-related disease, relative to the condition of the subject before administration of a compound described here or relative to a control population. For example, a reduction in frailty, an improvement in function in an age-related disability, the mitigation of a symptom of an age-related disease can be measured with reference to the pre-treatment condition of the subject or relative to a control population. Delay in onset of frailty, age-related disability, or age-related disease is typically measured with reference to a control population.

An improvement (i.e., a reduction) in frailty can be measured as increased strength, weight gain, faster mobility, increased energy, increased levels of activity, increased endurance, and/or enhanced behavioral response to a sensory cue. Alternatively or in addition, a decrease in one or more inflammatory biomarkers, an improvement in glucose homeostasis, and a decrease in one of more biomarkers of clotting activation can indicate a reduction in frailty.

The mitigation of a symptom of an age-related disease, such as osteoporosis, arthritis, cataracts, macular degeneration, and cardiovascular disease, can also indicate an improvement in a measure of life/health span in the methods described herein. For example, one or more cardiovascular parameters, such as cholesterol level, triglyceride level, high density lipoprotein level, and/or blood pressure can be measured as an indicator of life/health span.

In particular embodiments, an improvement in a measure of life/health span can be detected as a reduction in, a reversal of, or delay in onset of sarcopenia, relative to the condition of the subject before treatment or relative to a control population. More specifically, reduction and/or reversal of sarcopenia can be measured with reference to the pre-treatment condition of the subject or relative to a control population; whereas delay in onset of sarcopenia is typically measured with reference to a control population.

In certain embodiments, an improvement in a measure of life/health span can be detected as reduction in, a reversal of, or delay in onset of an age-related increase in lipofuscin accumulation, relative to the condition of the subject before administration of a compound described herein or relative to a control population. In particular, reduction and/or reversal of sarcopenia can be measured with reference to the pre-treatment condition of the subject or relative to a control population; whereas delay in onset of excess liposfuscin is typically measured with reference to a control population. Illustrative tissues in which in which lipofuscin accumulated and can be measured include brain, heart, liver, spleen, and kidney.

Alternatively or in addition, improved life/health span can be detected by detecting an enhanced ability to maintain homeostasis during the application of a stressor and/or a reduced time required to return to homeostasis after the application of a stressor. For example, responses to stressors including drug-induced oxidative stress, exposure to heat, and exposure to cold can be measured to determine whether the subject has an enhanced ability to maintain and/or return to homeostasis after being stressed.

In particular embodiments, the measure of life/health span includes the level and/or activity of a molecule that plays a role in protein trafficking, the autophagy pathway, ubiquitination, and/or lysozomal degradation of proteins. An example of the latter is lysosome-associated membrane protein-2 (LAMP-2).

Other indicators of life/health span can include the number of inclusion bodies in muscle tissue, and/or mitochondrial function and/or morphology.

D. Subjects

The methods described herein are typically carried out using subject who are suffering from, or determined to be at risk for a decline in a measure of life/health span. Thus for example, these methods can be performed on a subject suffering from, or determined to be at risk for, frailty, an age-related disability, or an age-related disease. In various embodiments, where the subject is suffering from, or determined to be at risk for, frailty, the subject is determined to have at least two, three, four, five, six, or seven symptoms selected: weakness, weight loss, slowed mobility, fatigue, low levels of activity, poor endurance, and impaired behavioral response to a sensory cue. Alternatively or in addition, the subject may have one or more symptoms selected from an increase in one or more inflammatory biomarkers, glucose homeostasis impairment, and an increase in one of more biomarkers of clotting activation.

In particular embodiments, the subject is suffering from sarcopenia and/or has lipofuscin accumulation in one or more of brain, heart, liver, spleen, and kidney. Alternatively or in addition, the subject may have a reduced ability to maintain homeostasis during the application of a stressor and/or may require an extended time required to return to homeostasis after the application of a stressor. In such embodiments, the reduced ability or extended time is relative to the condition of the subject at a previous time or relative to a normal ability or time.

In particular embodiments, the subject may display an abnormal level and/or activity of a molecule that plays a role in protein trafficking, the autophagy pathway, ubiquitination, and/or lysozomal degradation of proteins (e.g., LAMP-2). Alternatively or in addition, the subject may have abnormal inclusion bodies in muscle tissue and/or an abnormality in mitochondrial function and/or morphology. Such changes may be observed relative to the condition of the subject at a previous time or relative to a normal (e.g., non-aged adult) subject.

E. Co-Administration of Compounds with Additional Agents

In a particular embodiment of the method, a compound described herein (e.g., a thioflavin compound, or a derivative thereof) is co-administered with an additional agent that is useful for increasing life span and/or health span. In this embodiment, the amount of additional agent administered is sufficient to produce a beneficial effect in the subject when co-administered with the selected compound.

Any additional agent that increases life span and/or health span and is tolerated by the subject can be employed in a method of the invention. In particular embodiments, the additional agent is one that acts by a different mechanism than the compound with which it is co-administered. Examples of additional agents suitable for use these embodiments include compounds that mimic the catalytic activities of antioxidant enzymes (e.g., EUK-134, carboxyfullerenes) or that act as non-catalytic antioxidants (e.g., lipoic acid, trolox) and/or other compounds shown to extend lifespan in animal models including, but not limited to, rapamycin, metformin, valproic acid, ethosuximide, trimethadione, 3,3-diethyl-2-pyrrolidinone, lithium, and resveratrol and/or its derivatives.

F. Formulations

In order to carry out certain embodiments of the invention, one or more active agents (e.g., thioflavin compounds or derivatives thereof) described herein are administered, e.g., to an individual at risk for, or suffering from, frailty, an age-related disability, or an age-related disease.

The active agent(s) can be administered in the “native” form or, if desired, in the form of salts, esters, amides, prodrugs, derivatives, and the like, provided the salt, ester, amide, prodrug, or derivative is suitable pharmacologically, i.e., effective in the present method. Salts, esters, amides, prodrugs, and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Pharmaceutically acceptable salts of the compounds described herein include those derived from pharmaceutically acceptable, inorganic and organic acids and bases. Examples of suitable acids include hydrochloric, hydrobromic, sulfuric, nitric, perchloric, fumaric, maleic, phosphoric, glycollic, lactic, salicyclic, succinic, gluconic, isethionic, glycinic, malic, mucoic, glutammic, sulphamic, ascorbic acid; toluene-p-sulfonic, tartaric, acetic, citric, methanesulfonic, formic, benzoic, malonic, naphthalene-2-sulfonic, trifluoroacetic and benzenesulfonic acids. Salts derived from appropriate bases include, but are not limited to alkali such as sodium and ammonium.

For example, acid addition salts are prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Illustrative acid addition salts of the active agents herein are halide salts, such as may be prepared using hydrochloric or hydrobromic acids. Conversely, basic salts of the active agents described herein are prepared in a similar manner using a pharmaceutically acceptable base such as sodium hydroxide, potassium hydroxide, ammonium hydroxide, calcium hydroxide, trimethylamine, or the like. Illustrative basic salts include alkali metal salts, e.g., the sodium salt, and copper salts.

Acid addition salts useful in the methods described herein include the physiologically compatible acid addition salts, most preferably the dihydrochloride. Bis-quaternary salts useful in the methods described herein include the physiologically compatible bis-quaternary salts, such as the methiodide and the dimethiodide.

Preparation of Esters Typically Involves Functionalization of Hydroxyl and/or carboxyl groups and/or other reactive groups that may be present within the molecular structure of the drug. The esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides and prodrugs can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety that results in a compound that is therapeutically inactive until modified by an individual's metabolic system.

The active agents described herein are typically combined with a pharmaceutically acceptable carrier (excipient), such as are described in Remington's Pharmaceutical Sciences (1980) 16th editions, Osol, ed., 1980. Pharmaceutically acceptable carriers can contain one or more physiologically acceptable compound(s) that act, for example, to stabilize the composition or to increase or decrease the absorption of the active agent(s). A pharmaceutically acceptable carrier suitable for use in the methods described herein is non-toxic to cells, tissues, or subjects at the dosages employed, and can include a buffer (such as a phosphate buffer, citrate buffer, and buffers made from other organic acids), an antioxidant (e.g., ascorbic acid), a low-molecular weight (less than about 10 residues) peptide, a polypeptide (such as serum albumin, gelatin, and an immunoglobulin), a hydrophilic polymer (such as polyvinylpyrrolidone), an amino acid (such as glycine, glutamine, asparagine, arginine, and/or lysine), a monosaccharide, a disaccharide, and/or other carbohydrates (including glucose, mannose, and dextrins), a chelating agent (e.g., ethylenediaminetetratacetic acid [EDTA]), a sugar alcohol (such as mannitol and sorbitol), a salt-forming counterion (e.g., sodium), and/or an anionic surfactant (such as Tween™, Pluronics™, and PEG). In one embodiment, the pharmaceutically acceptable carrier is an aqueous pH-buffered solution.

Other pharmaceutically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

Pharmaceutical compositions described herein can be stored in any standard form, including, e.g., an aqueous solution or a lyophilized cake. Such compositions are typically sterile when administered to subjects. Sterilization of an aqueous solution is readily accomplished by filtration through a sterile filtration membrane. If the composition is stored in lyophilized form, the composition can be filtered before or after lyophilization and reconstitution.

When active agents described herein contain chiral or prochiral centres they can exist in different stereoisomeric forms including enantiomers of (+) and (−) type or mixtures of them. The present invention includes in its scope both the individual isomers and the mixtures thereof.

It will be understood that, when mixtures of optical isomers are present, they may be separated according to the classic resolution methods based on their different physicochemical properties, e.g. by fractional crystallization of their acid addition salts with a suitable optically active acid or by the chromatographic separation with a suitable mixture of solvents.

G. Administration

The active agents identified herein are useful for intravenous, intraarterial, intrathecal, intradermal, intracavitary, oral, rectal, intramuscular, subcutaneous, intracisternal, intravaginal, intraperitonial, topical, buccal, and nasal administration to improve life/health span. In various embodiments, the active agents described herein can be administered orally, in which case delivery can be enhanced by the use of protective excipients. This is typically accomplished either by complexing the active agent(s) with a composition to render them resistant to acidic and enzymatic hydrolysis or by packaging the agents in an appropriately resistant carrier, e.g. a liposome. Means of protecting agents for oral delivery are well known in the art (see, e.g., U.S. Pat. No. 5,391,377).

Elevated serum half-life can be maintained by the use of sustained-release “packaging” systems. Such sustained release systems are well known to those of skill in the art (see, e.g., Tracy (1998) Biotechnol. Prog. 14: 108; Johnson et al. (1996), Nature Med. 2: 795; Herbert et al. (1998), Pharmaceut. Res. 15, 357).

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, lipid complexes, etc. In another embodiment, one or more components of a solution can be provided as a “concentrate,” e.g., in a storage container (e.g., in a premeasured volume) ready for dilution or in a soluble capsule ready for addition to a volume of water.

Other illustrative formulations for topical delivery include, but are not limited to, ointments and creams. Ointments are semisolid preparations, which are typically based on petrolatum or other petroleum derivatives. Creams containing the selected active agent, are typically viscous liquid or semisolid emulsions, often either oil-in-water or water-in-oil. Cream bases are typically water-washable and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base is preferably inert, stable, nonirritating, and nonsensitizing.

In certain embodiments, the agents may also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

In certain embodiments, one or more active agents described herein are administered alone or in combination with other therapeutics in implantable (e.g., subcutaneous) matrices, termed “depot formulations.”

A major problem with standard drug dosing is that typical delivery of drugs results in a quick burst of medication at the time of dosing, followed by a rapid loss of the drug from the body. Most of the side effects of a drug occur during the burst phase of its release into the bloodstream. Secondly, the time the drug is in the bloodstream at therapeutic levels is very short; most is used and cleared during the short burst.

Drugs (e.g., the active agents described herein) imbedded in various matrix materials for sustained release can mitigate these problems. Drugs embedded, for example, in polymer beads or in polymer wafers have several advantages. First, most systems allow slow release of the drug, thus creating a continuous dosing of the body with small levels of drug. This typically prevents side effects associated with high burst levels of normal injected or pill-based drugs. Secondly, since these polymers can be made to release over hours to months, the therapeutic span of the drug is markedly increased. Often, by mixing different ratios of the same polymer components, polymers of different degradation rates can be made, allowing remarkable flexibility depending on the agent being used. A long rate of drug release is beneficial for people who might have trouble staying on regular dosage, such as the elderly, but also represents an ease of use improvement that everyone can appreciate. Most polymers can be made to degrade and be cleared by the body over time, so they will not remain in the body after the therapeutic interval.

Another advantage of polymer-based drug delivery is that the polymers often can stabilize or solubilize proteins, peptides, and other large molecules that would otherwise be unusable as medications. Finally, many drug/polymer mixes can be placed directly in the disease area, allowing specific targeting of the medication where it is needed without losing drug to the “first pass” effect. This is certainly effective for treating the brain, which is often deprived of medicines that can't penetrate the blood/brain barrier.

A wide variety of approaches to designing depot formulations that provide sustained release of an active agent are known and are suitable for use in the methods described herein. Generally, the components of such formulations are biocompatible and may be biodegradable. Biocompatible polymeric materials have been used extensively in therapeutic drug delivery and medical implant applications to effect a localized and sustained release. See Leong et al., “Polymeric Controlled Drug Delivery,” Advanced Drug Delivery Rev., 1:199-233 (1987); Langer, “New Methods of Drug Delivery,” Science, 249:1527-33 (1990); Chien et al., Novel Drug Delivery Systems (1982). Such delivery systems offer the potential of enhanced therapeutic efficacy and reduced overall toxicity.

Examples of classes of synthetic polymers that have been studied as possible solid biodegradable materials include polyesters (Pitt et al., “Biodegradable Drug Delivery Systems Based on Aliphatic Polyesters: Applications to Contraceptives and Narcotic Antagonists,” Controlled Release of Bioactive Materials, 19-44 (Richard Baker ed., 1980); poly(amino acids) and pseudo-poly(amino acids) (Pulapura et al. “Trends in the Development of Bioresorbable Polymers for Medical Applications,” J. Biomaterials Appl., 6:1, 216-50 (1992); polyurethanes (Bruin et al., “Biodegradable Lysine Diisocyanate-based Poly(Glycolide-co-.epsilon. Caprolactone)-Urethane Network in Artificial Skin,” Biomaterials, 11:4, 291-95 (1990); polyorthoesters (Heller et al., “Release of Norethindrone from Poly(Ortho Esters),” Polymer Engineering Sci., 21:11, 727-31 (1981); and polyanhydrides (Leong et al., “Polyanhydrides for Controlled Release of Bioactive Agents,” Biomaterials 7:5, 364-71 (1986).

Thus, for example, the active agent(s) can be incorporated into a biocompatible polymeric composition and formed into the desired shape outside the body. This solid implant is then typically inserted into the body of the subject through an incision. Alternatively, small discrete particles composed of these polymeric compositions can be injected into the body, e.g., using a syringe. In an illustrative embodiment, the active agent(s) can be encapsulated in microspheres of poly (D,L-lactide) polymer suspended in a diluent of water, mannitol, carboxymethyl-cellulose, and polysorbate 80. The polylactide polymer is gradually metabolized to carbon dioxide and water, releasing the active agent(s) into the system.

In yet another approach, depot formulations can be injected via syringe as a liquid polymeric composition. Liquid polymeric compositions useful for biodegradable controlled release drug delivery systems are described, e.g., in U.S. Pat. Nos. 4,938,763; 5,702,716; 5,744,153; 5,990,194; and 5,324,519. After injection in a liquid state or, alternatively, as a solution, the composition coagulates into a solid.

One type of polymeric composition suitable for this application includes a nonreactive thermoplastic polymer or copolymer dissolved in a body fluid-dispersible solvent. This polymeric solution is placed into the body where the polymer congeals or precipitates and solidifies upon the dissipation or diffusion of the solvent into the surrounding body tissues. See, e.g., Dunn et al., U.S. Pat. Nos. 5,278,201; 5,278,202; and 5,340,849 (disclosing a thermoplastic drug delivery system in which a solid, linear-chain, biodegradable polymer or copolymer is dissolved in a solvent to form a liquid solution).

The active agent(s) can also be adsorbed onto a membrane, such as a silastic membrane, which can be implanted, as described in International Publication No. WO 91/04014. Other illustrative implantable sustained release systems include, but are not limited to Re-Gel®, SQ2Gel®, and Oligosphere® by MacroMed, ProLease® and Medisorb® by Alkermes, Paclimer® and Gliadel® Wafer by Guilford pharmaceuticals, the Duros implant by Alza, acoustic biSpheres by Point Biomedical, the Intelsite capsule by Scintipharma, Inc., and the like.

The compounds described herein can be co-administered with additional agents that improve life/health span by simultaneous administration or sequential administration. In the case of sequential administration, the first administered agent must be exerting some physiological effect on the subject when the second agent is administered or becomes active in the subject.

Additional agents can be administered by a route that is the same as, or different from, the route of administration of the compounds described herein (e.g., thioflavin compound or derivatives thereof). Where possible, it is generally desirable to administer these agents by the same route of administration, preferably in the same formulation. However, differences in pharmacodynamics, pharmacokinetics, or other considerations may dictate the co-administration of selected compound and additional agent in separate formulations. Additional agents can be administered according to standard practice.

H. Dose

In therapeutic applications, the compositions described herein are administered to a subject in an amount sufficient to improve at least one measure of life/health span. Amounts effective for this use will depend upon the status of the measure of life/health span, the degree of improvement sought, and the general state of the subject's health. Single or multiple administrations of the compositions may be administered depending on the dosage and frequency as required and tolerated by the subject.

The concentration of active agent(s) can vary widely and will be selected primarily based on fluid volumes, viscosities, body weight and the like in accordance with the particular mode of administration selected and the subject's needs. In accordance with standard practice, the clinician can titer the dosage and modify the route of administration as required to obtain the optimal therapeutic effect. Generally, the clinician begins with a low dose and increases the dosage until the desired therapeutic effect is achieved. Starting doses for a given active agent can, for example be extrapolated from in vitro and/or animal data.

In particular embodiments, concentrations of active agent(s) will typically be selected to provide dosages ranging from about 0.0001 μg/kg/day to about 10 mg/kg/day and sometimes higher. Typical dosages range from about 0.001 μg/kg/day to about 1 mg/kg/day, specifically from about 0.01 μg/kg/day to about 100 μg/kg/clay, more specifically from about 0.1 μg/kg/day to about 10 μg/kg/day, e.g., about 1 μg/kg/day. It will be appreciated that such dosages may be varied to optimize a therapeutic regimen in a particular subject or group of subjects, and thus any of these values can represent the upper or lower limit of a suitable dosage range according to the invention (e.g., about 0.001 μg/kg to about 10 μg/kg).

In embodiments of the method in which an additional agent that improves life/health span is co-administered with a compound described herein, suitable doses of additional agents are known and can be adjusted by the clinician for co-administration with a thioflavin compound or derivative.

### Pharmaceutical Compositions

The invention provides pharmaceutical compositions useful in one or more of the above-described methods. In one embodiment, the pharmaceutical composition includes a selected compound (e.g., athioflavin compound, or a derivative thereof), an additional agent that is useful for improving life/health span, and a pharmaceutically acceptable carrier in a single composition. The selection of thioflavins or thioflavin derivatives, additional agents, and carriers for such compositions are as described above.

In another embodiment, the pharmaceutical composition includes a selected compound (e.g., athioflavin compound, or a derivative thereof), according to the invention and a pharmaceutically acceptable carrier, incorporated into a transdermal patch or depot formulation. Suitable carriers, patches, and depot formulations are described above. In a particular embodiment, the patch or depot formulation includes an additional agent that improves life/health span, as described above.

It is understood that the examples and embodiments described herein are for illustrative purposes only and that various modifications or changes in light thereof will be suggested to persons skilled in the art and are to be included within the spirit and purview of this application and scope of the appended claims.

In addition, all other publications, patents, and patent applications cited herein are hereby incorporated by reference in their entirety for all purposes.

### EXAMPLE

The following example is offered to illustrate, but not to limit, the claimed invention.

### Example 1

### Amyloid-Binding Compounds Maintain Protein Homeostasis During Aging and Extend Lifespan

### Abstract

A group of small molecules, traditionally employed in histopathology to stain amyloids in tissues, not only bind protein fibrils but slow aggregation in vitro and in cell culture^{3 4}. This study was based on the hypothesis that treating animals with such compounds would promote proteostasis in vivo and increase longevity. Here it was found that exposure of adult Caenorhabditis elegans to the amyloid-binding dye Thioflavin T (ThT) resulted in a profoundly extended lifespan and slowed ageing. ThT also suppressed pathological features of mutant metastable proteins and human β-amyloid-associated toxicity. These beneficial effects of ThT depend on the proteostasis network regulator Heat Shock Factor 1 (HSF-1), molecular chaperones, autophagy and proteosomal functions. These results demonstrate that pharmacologic maintenance of the proteostatic network has a profound impact in ageing rates and this prompts the development of novel therapeutic interventions against ageing and age-related diseases.

### Results and Discussion

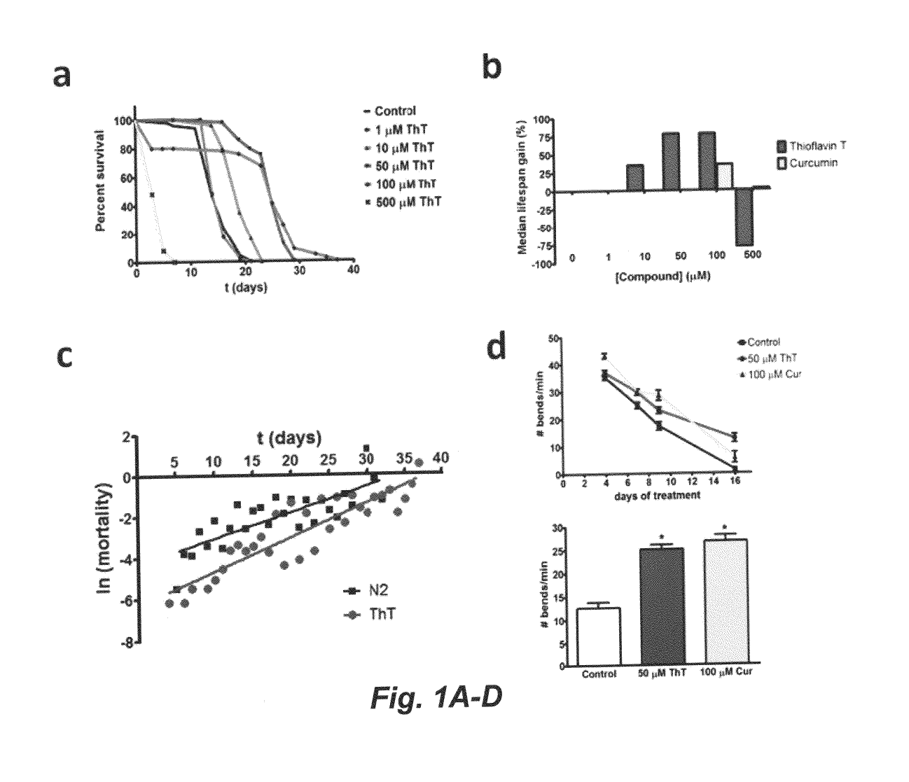

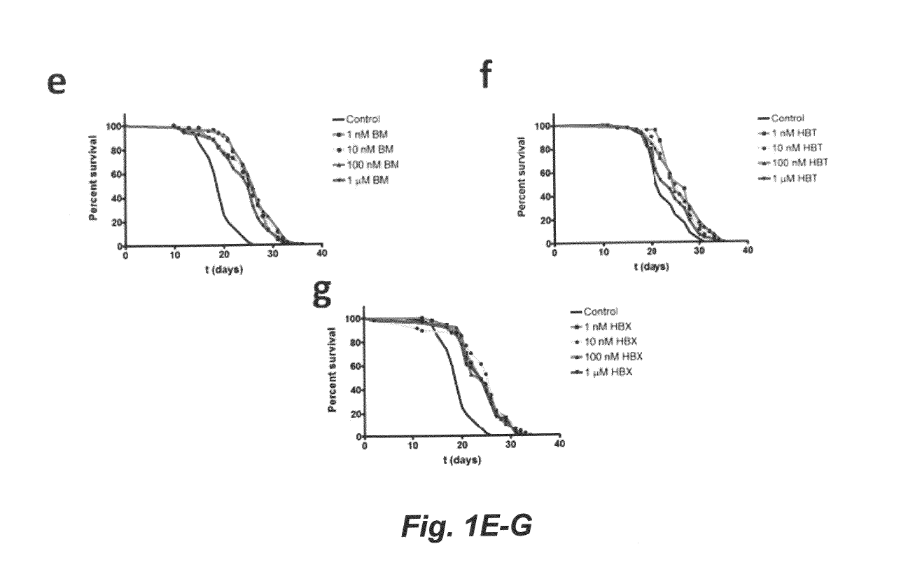

The longevity of the nematode Caenorabditis elegans is influenced by hundreds of genes including an insulin-like signaling pathway (ILS) that regulates the activities of the FOXO-like transcription factor DAF-16⁵ and the Nrf2-like transcription factor SKN-1⁶. Together with the stress response transcription factor Heat Shock Factor 1 (HSF-1), DAF-16 regulates protein homeostasis (proteostasis) in C. elegans and modulates lifespan^{7,8,9}. To test the hypothesis that chemical compounds that exhibit protein fibril- and protein aggregate-binding properties may influence the course of age-related protein aggregate formation, a series of such compounds was tested for effects on longevity of C. elegans. Exposing sterile wildtype (N2) worms to the fibril-binding flavonoid Thioflavin-T (4-(3,6-dimethyl-1,3-benzothiazol-3-ium-2-yl)-N,N-dimethylaniline chloride; ThT)¹⁰ at either 50 or 100 μM throughout adult life leads to a reproducible and significant increase in median (60%) and maximal lifespan (43-78%; FIG. 1a, Supplementary Table 1).

SUPPLEMENTARY TABLE 1
Summary of survival analysis of N2 worms exposed to ThT.

| | Median LS | Median LS | Median | Log-rank |
|---|---|---|---|---|
| Experiment | (Control) | (ThT) | increase (%) | P values |
| I | 16 | 23.5 | 46.9 | P < 0.0001 |
| II | 14 | 25 | 78.6 | P < 0.0001 |
| III | 16 | 23 | 43.7 | P < 0.0001 |
| IV | 13 | 21 | 61.5 | P < 0.0001 |
| V | 15 | 23 | 53.3 | P < 0.0001 |
| VI | 20 | 31 | 55 | P < 0.0001 |
| VII | 18 | 31 | 72.2 | P < 0.0001 |
| VII | 18.5 | 29 | 56.8 | P < 0.0001 |
| IX | 10 | 17 | 70.0 | P < 0.0001 |
| X | 13 | 21 | 61.5 | P < 0.0001 |

Log-rank P values from comparisons between treated (ThT 50 μM) and untreated control worms.

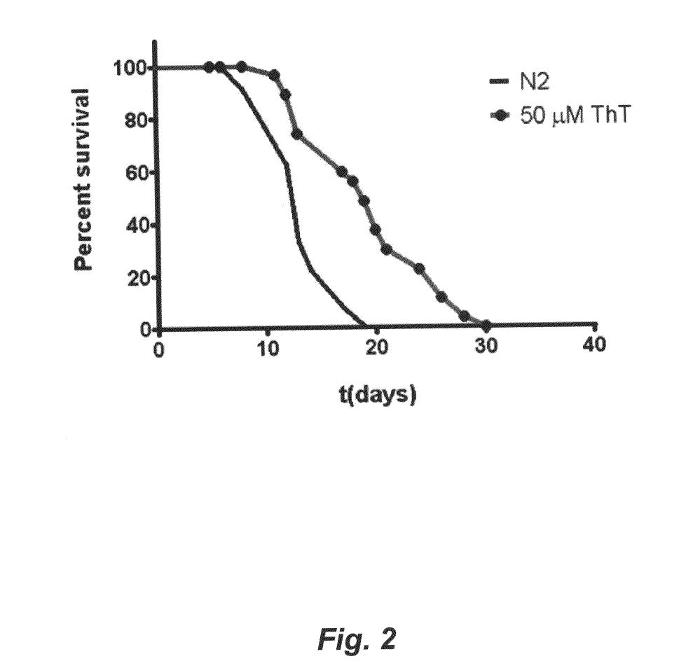

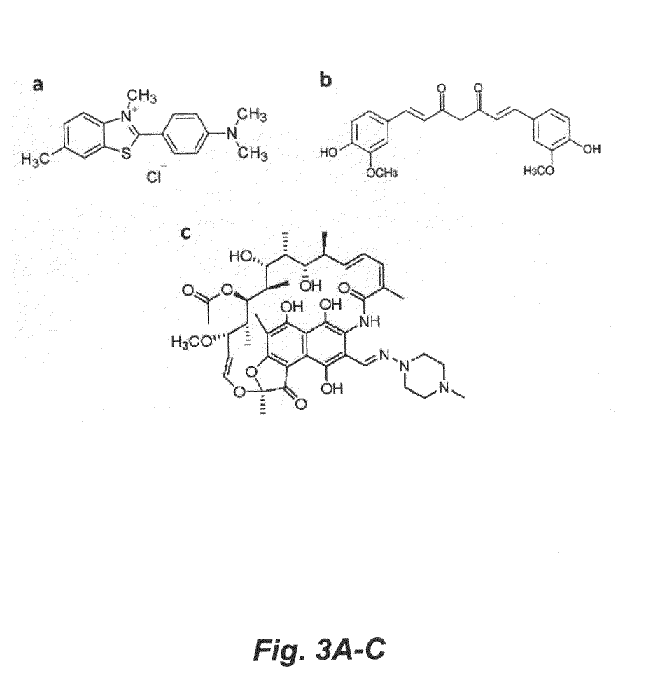

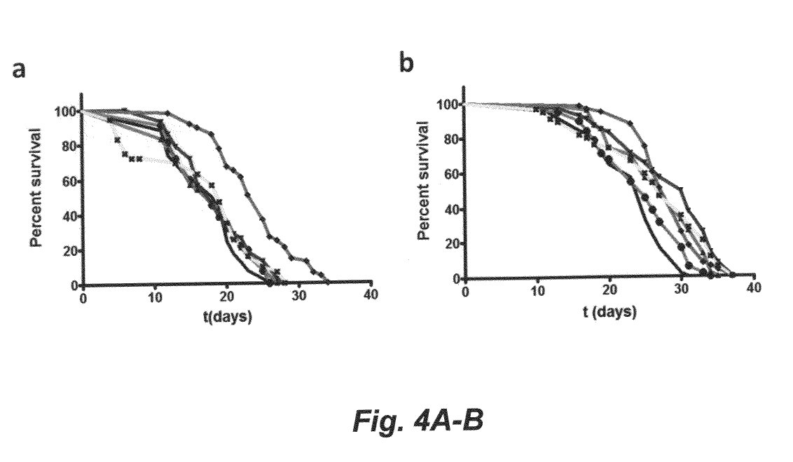

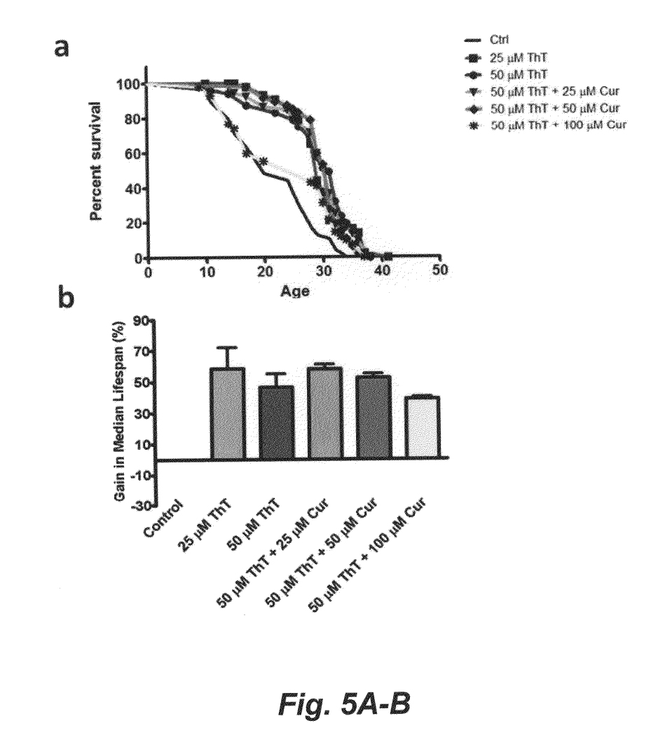

Similar results were obtained with fertile worms (FIG. 2). The compound reduced age-specific mortality at all ages (p<0.001, FIG. 1c) and retarded age-related decline in spontaneous movement (FIG. 1d) consistent with improvements in health throughout the adulthood of ThT-treated worms. However, at higher doses (500 μM) ThT appears toxic and significantly shortens lifespan (FIGS. 1a, b). Other compounds with protein aggregate-binding properties, including curcumin and rifampicin, increased lifespan to a lesser extent (up to 45%) (FIGS. 3, 4). When ThT and curcumin treatments were combined, we did not observe additive effects on lifespan (FIG. 5).

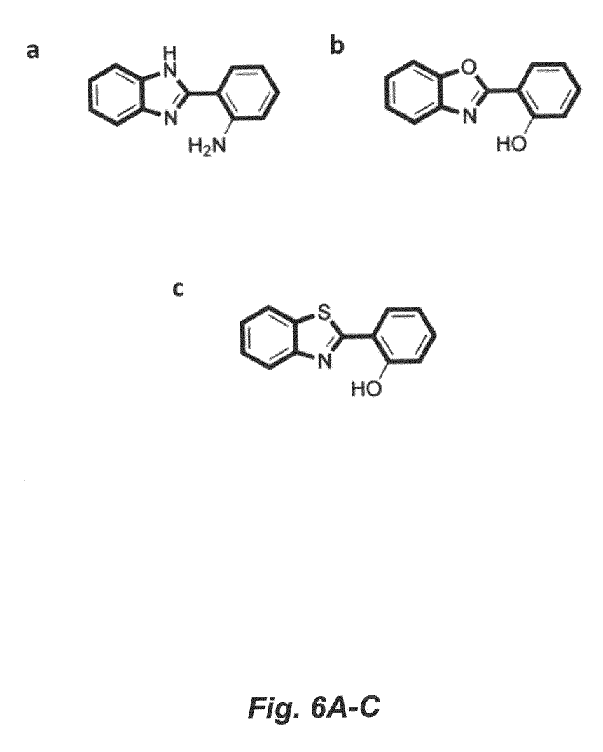

We then tested several compounds with similar structural features to ThT, but with different pharmacological properties: 2-(2-hydroxyphenyl)-benzoxazole (HBT), 2-(2-hydroxyphenyl)benzothiazole (HBX) and 2-(2-aminophenyl)-1H-benzimidazole (BM) (FIG. 6). These compounds also extended the lifespan of adult worms (up to 40%) but at concentrations significantly lower than ThT (FIGS. 1e, f, g) suggesting that the bioavailability of ThT-like compounds is an important influence on lifespan.

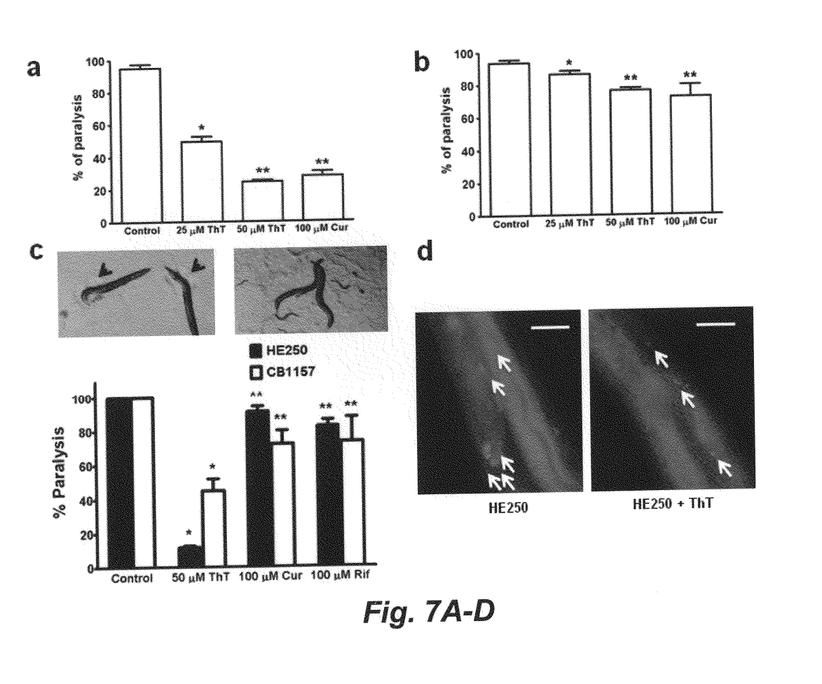

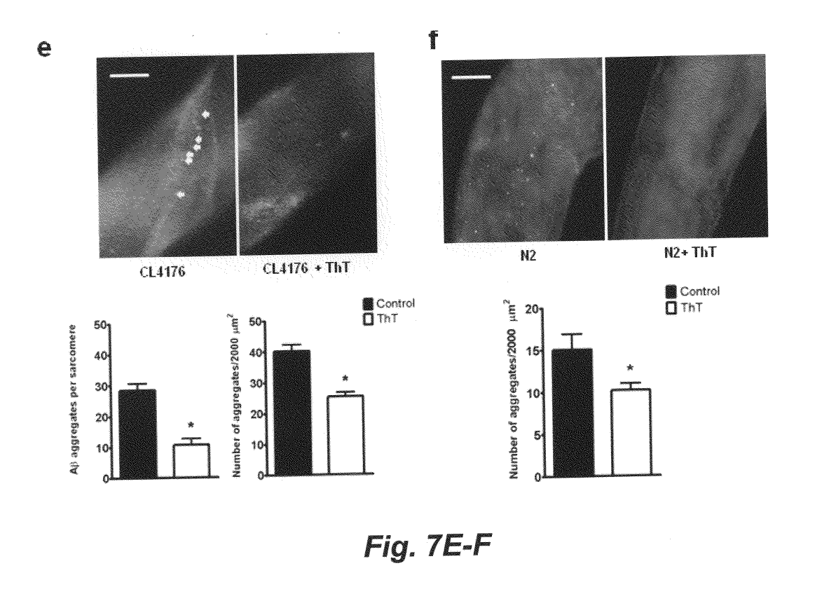

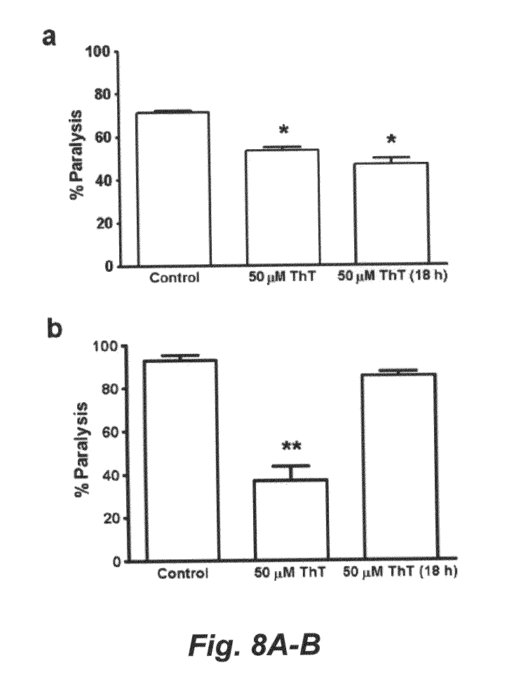

To test the effects of ThT on proteostasis two C. elegans models of human proteotoxicity disease were used: the strain CL4176 dvIs27[myo-3::Aβ₃₋₄₂ let 3′UTR(pAF29); pRF4 (rol-6(su1006))]¹¹, which expresses an aggregating Aβ peptide₃₋₄₂ in muscle tissue¹² and AM140 (rmIs132[P(unc-54) Q35::YFP]) which expresses a polyglutamine (polyQ) expansion protein. Aβ aggregates are associated with pathological lesions in Alzheimer's disease (AD) whilst polyQ aggregation is a feature of a number of neurological conditions¹³. When raised at 25° C., worms expressing these proteins in muscle accumulate aggregates of heterologous proteins and develop paralysis. We found that 50 μM ThT and 100 μM curcumin significantly decrease the proportion of paralyzed worms (FIGS. 7a, b). We examined Aβ aggregation in vivo by immunohistochemistry and found that ThT reduced aggregate formation and preserved the muscle integrity in CL4176 (FIG. 7e). We also found that ThT rescued the Aβ₃₋₄₂ aggregation-induced paralysis even when worms were treated 18 h after the induction of aggregate formation, suggesting that ThT can ameliorate detrimental effects during the course of the aggregate-related pathology (FIG. 8).

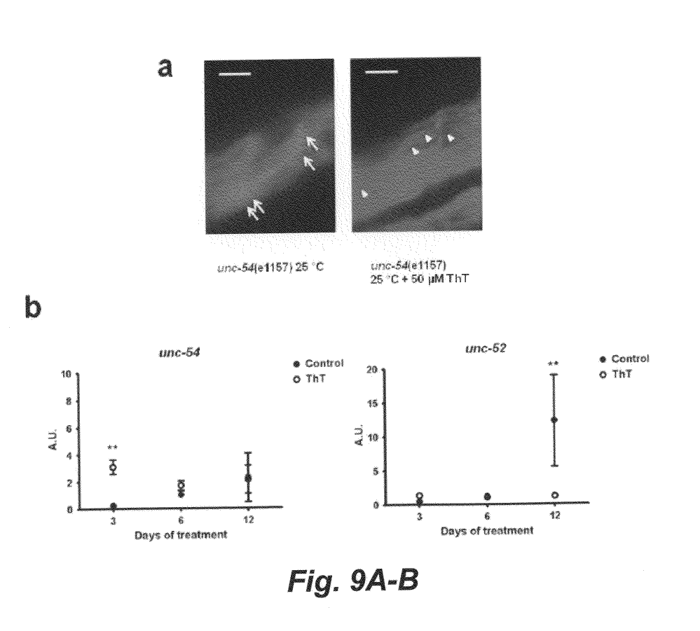

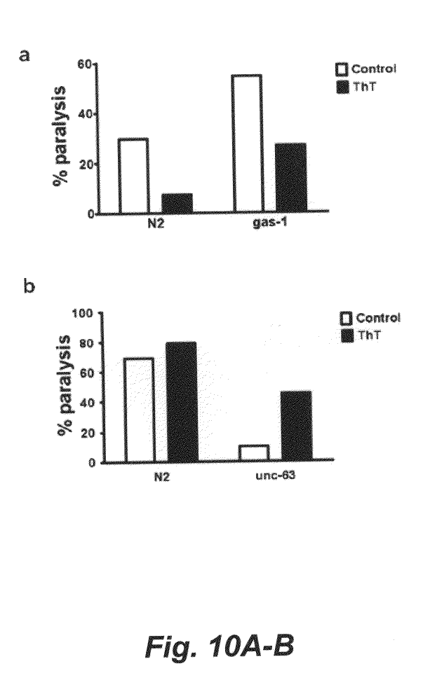

If amyloid-binding compounds extended lifespan through improved proteostasis, then it was expected that they would influence not only heterologous disease-related models, but also worm proteins. ThT and curcumin were tested on mutant worms that express metastable worm proteins previously exploited as indicators of the status of the proteostasis network capacity¹⁴. Strains carrying mutations in the gene unc-52 (HE250 [unc-52(e669su250)II]) or unc-54 (CB1157 [unc-54(e1157)I]) produce temperature-sensitive (ts) muscle proteins UNC-52 (perlecan) and UNC-54 (paramyosin), respectively, that exhibit altered structure and function^{15,16}. When switched to the restrictive temperature (25° C.) these worms become paralyzed¹⁴. We found that ThT suppressed paralysis of unc-52 and unc-54 mutants (FIGS. 7c, d), prevented the disruption of the muscle sarcomeres (FIG. 9) and restored perlecan organization (FIG. 7c). These observations were extended to other ts missense protein folding mutations expressed in the neuromuscular junction and in the nervous system¹⁷. ThT suppressed ethanol sensitivity and levamisole resistance in a strain carrying the gas-1 (fc21) mutation in a subunit of mitochondrial complex I and in a strain carrying unc-63(x26), an alpha subunit of the nicotinic acetylcholine receptor (FIG. 10) demonstrating that ThT acts in different tissues.

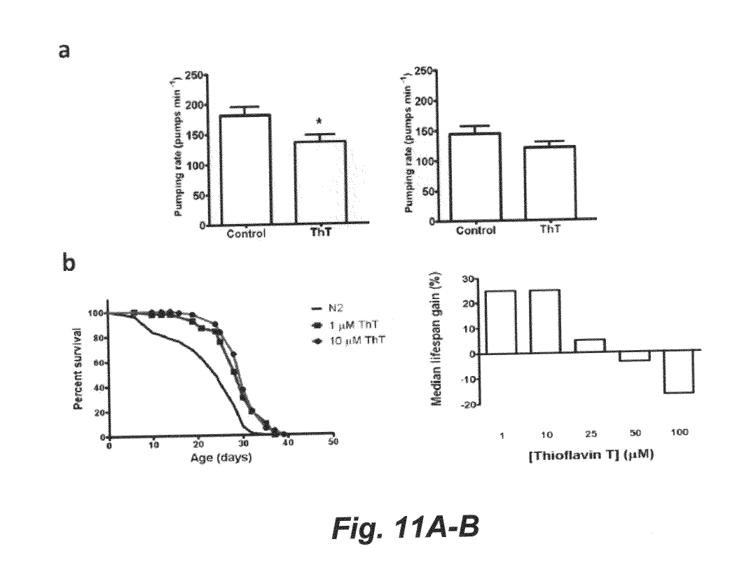

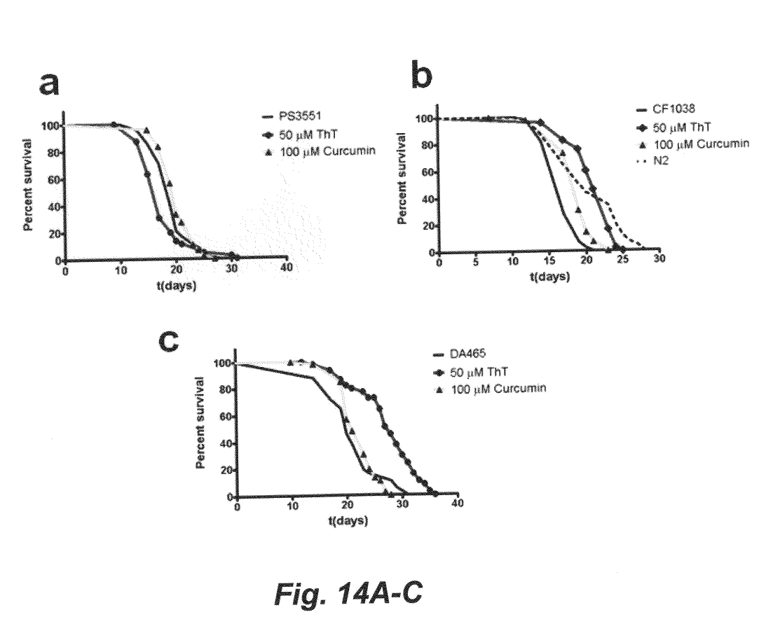

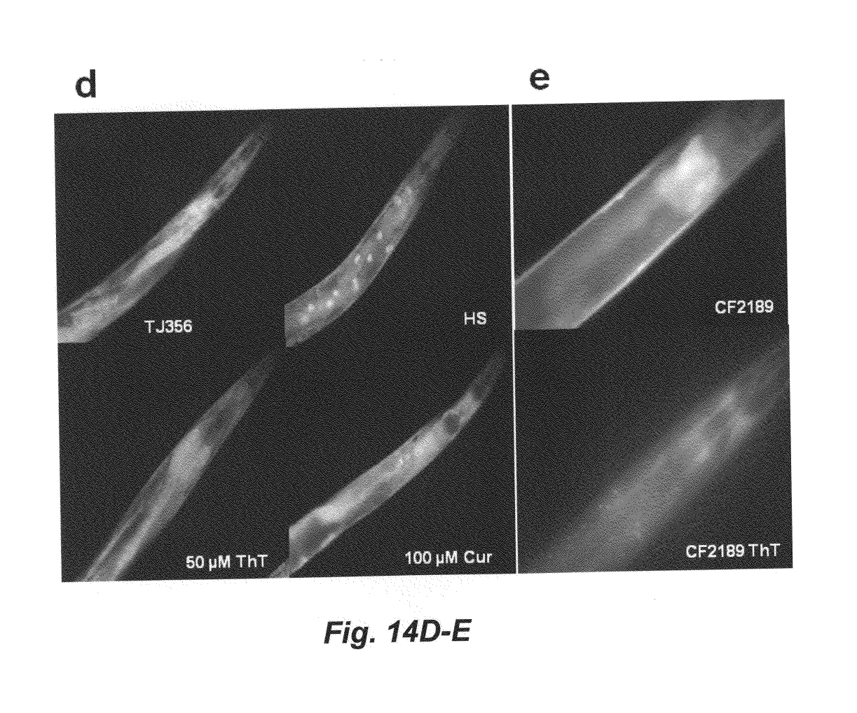

Since certain forms of dietary restriction (DR) suppress protein aggregation and increase lifespan, we asked whether ThT was acting as a DR mimetic. We observed that ThT produces a small decrease in pharyngeal pumping rate (˜15%) after 3 days of treatment, which could slightly decrease food intake but would be insufficient for the major lifespan extension we observe with the ThT treatment. No difference was detected after 6 days of ThT treatment (FIG. 11a). ThT also increased the lifespan of a strain carrying the eat-2(ad1116) mutation (FIG. 14c), which causes a defect in pharyngeal pumping, thereby inducing a DR lifespan extension¹⁸. Dilution of the bacterial food source also leads lifespan extension by DR¹⁹. 50 μM ThT was detrimental to lifespan in this model of DR but lower concentrations of the compound, 1 and 10 μM, increased lifespan by 24% (FIG. 11b). Since ThT increases lifespan in both genetic and nutrient models of DR, ThT-induced lifespan extension is at least partially independent from DR.

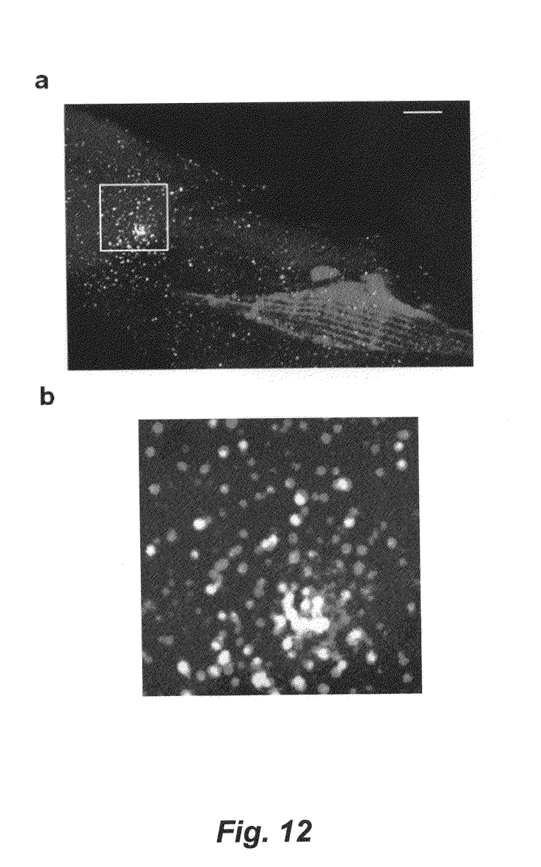

To determine whether ThT was interacting more directly with homeostatic mechanisms, in vivo ThT visualization was combined with Aβ₃₋₄₂ immunolocalization to show co-distribution of ThT with aggregates. To further investigate if ThT was influencing in vivo protein aggregation, the presence of amino acid sequence-independent oligomers of protein or peptides prone to aggregation was determined. Accumulated material was detected by an antibody specific for such oligomers (A11), during normal ageing, that was significantly decreased in both CL4176 and N2 by ThT treatment (FIG. 70. Also, the A11-positive signal was surrounded by and overlapping with ThT fluorescence (FIG. 12), suggesting a direct interaction between ThT and proteins prone to aggregation in vivo.

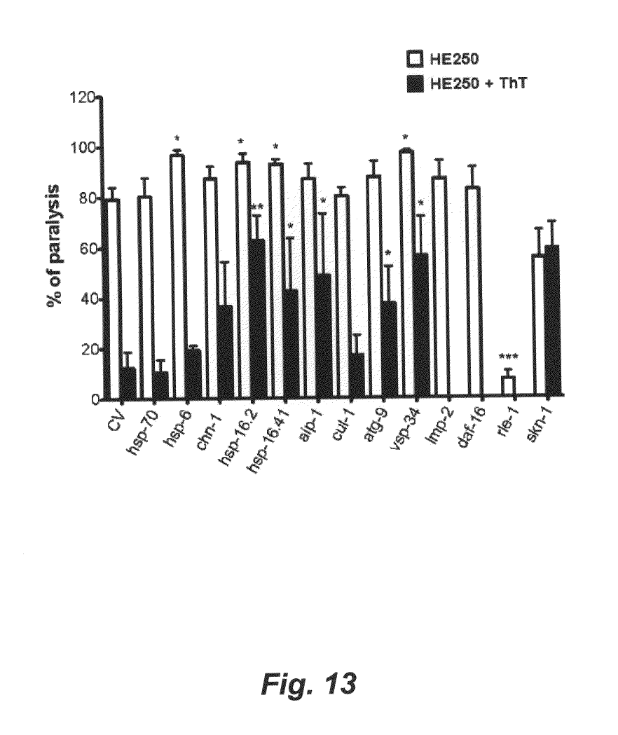

As DAF-16 and HSF-1 influence protein aggregation and lifespan through a number of downstream effectors involved in proteostasis^{8,20}, it was possible that ThT might also require components of this homeostatic network to suppress paralysis in the proteotoxic models. A targeted pharmacogenetic RNA interference (RNAi) screen was carried out of genes encoding several components of the ubiquitin/proteasome system, autophagy/lysosomal machinery and molecular chaperones. The first question was whether reducing the expression of genes encoding these proteins modulated the paralysis of metastable paramyosin mutants (FIG. 13). RNAi of small chaperones known to positively modulate lifespan in C. elegans, HSP-16.2 and HSP-16.4^{8,21}, and the mitochondrial HSP-70 (hsp-6) increased paralysis of HE250 mutants; however, RNAi of at least one of the multiple cytosolic HSP-70 genes (hsp-70) had no effect. An autophagy gene (vps-34) also influenced the HE250 mutant paralysis phenotype (FIG. 13). Interestingly, RNAi targeting rle-1, an E3 ubiquitin ligase that influences lifespan in C. elegans by determining the rate of DAF-16 degradation²², produced a remarkable reduction in the paralysis phenotype. This led us to test daf-16 (RNAi) but no change in the paralysis phenotype was observed suggesting that other proteins regulated by rle-1 can influence protein homeostasis.

Then, possible interactions between these protein homeostasis factors and the protective effect elicited by ThT on the HE250 paralysis phenotype was examined. ThT protection was decreased when combined with RNAi for several acute stress genes (e.g., hsp-16.2, hsp-16.41) consistent with a concerted action between chaperones and ThT to maintain protein conformation. Similarly, ubquitin/proteasome (aip-1) and autophagy/lysosomal (atg-9 and vps-34) functions were required for the beneficial effects of ThT. Interestingly, lmp-2, a protein involved in lysosome function significantly improved the ThT effect on paralysis. Since lmp-2 knockdown itself has no effect on paralysis it is possible that ThT is cleared from the cell by a lysosomal mechanism such that lmp-2(RNAi) results in increased ThT bioactivity.

Next, the dependency of ThT action on DAF-16 was explored. The ThT suppression of the paralysis phenotype was potentiated by daf-16 (RNAi) suggesting that some proteins activated by DAF-16 interfere with the mechanism elicited by ThT to promote proteostasis. This is consistent with the previous report of a reduction of Aβ aggregation by daf-16(RNAi)⁹. In contrast, skn-1 (RNAi), a transcription factor that positively modulates stress resistance and longevity, was required for ThT effect on the paralysis phenotype (FIG. 13), suggesting some SKN-1 target genes influence ThT action. However, no nuclear accumulation of a SKN-1 fusion protein [CF2189 (skn-1::GFP)] was observed, suggesting that there is no chronic stress during ThT treatment (FIG. 14e).

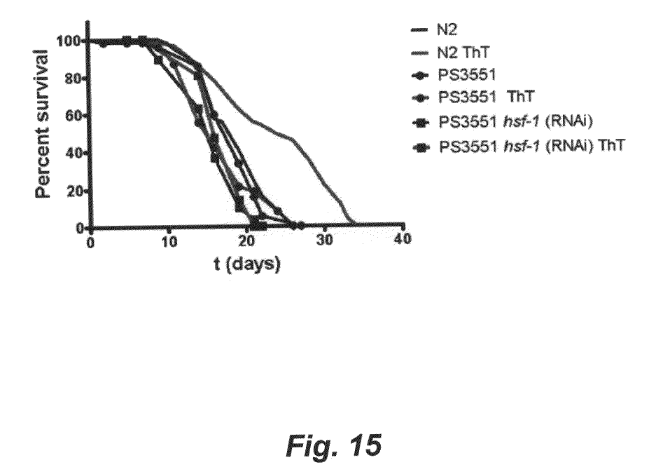

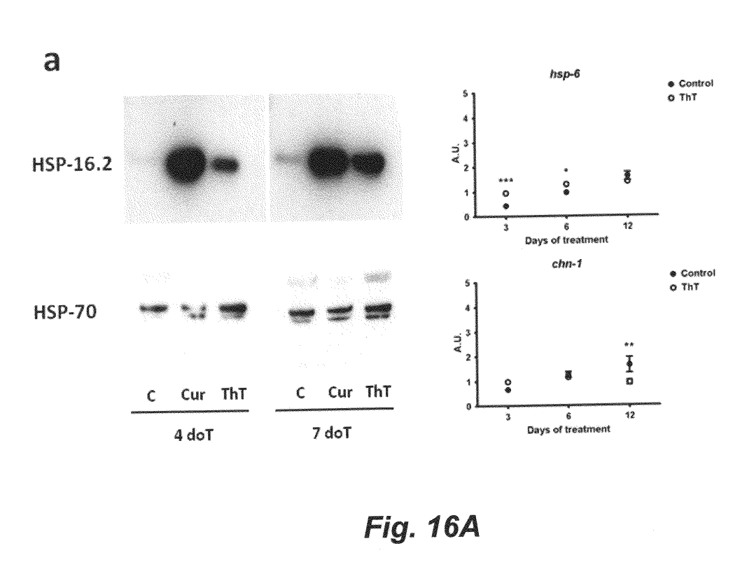

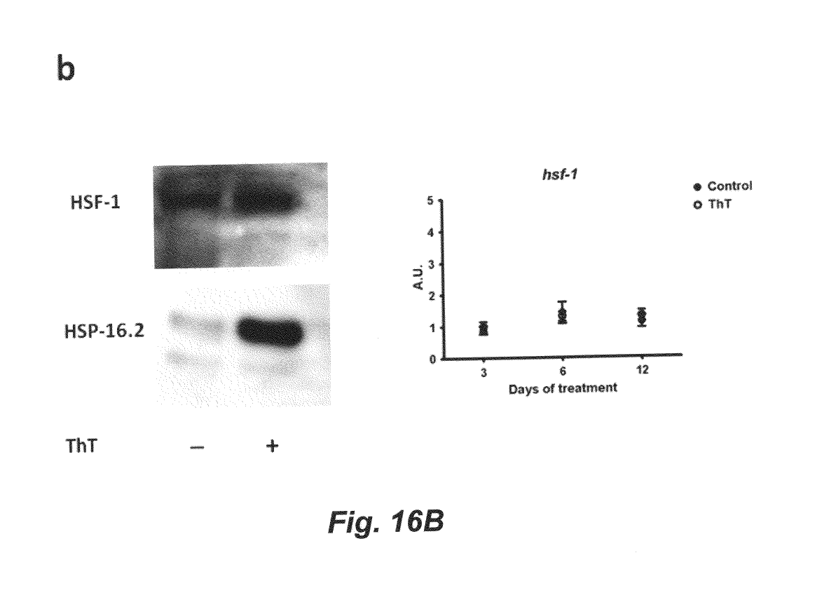

Returning to the question of whether ThT was extending lifespan by a similar mechanism, the transcription factor genes, hsf-1 and daf-16 was studied; mutation of either one shortens normal lifespan and suppresses the beneficial effects of a daf-2 mutation on the Aβ-aggregation model in C. elegans^{9,23}. ThT does not increase lifespan in a strain carrying the hsf-1(sy441) mutation which produces a non-functional HSF protein (FIG. 14a, FIG. 15) suggesting that the ThT effect on lifespan extension requires the active participation of the HSF-1-regulated machinery. Consistent with this idea, it was found that the protein levels of HSP-16.2 and HSP-70, two main contributors to the stress response, and the RNA levels of a mitochondrial (hsp-6) and a cytosolic (chn-1) hsp-70 isoform are up-regulated by ThT treatment (Supplementary FIG. 11a). We also detected a slight increase in the levels of HSF-1 protein under ThT treatment (Supplementary FIG. 11b).

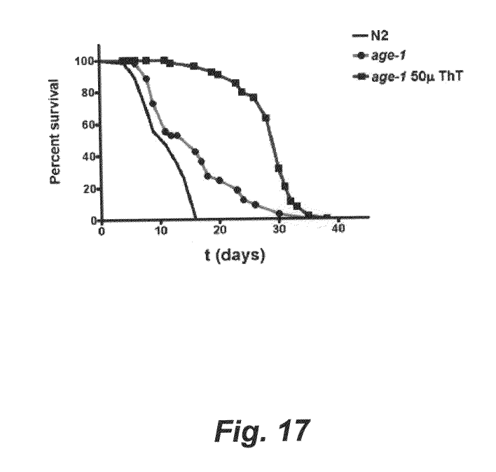

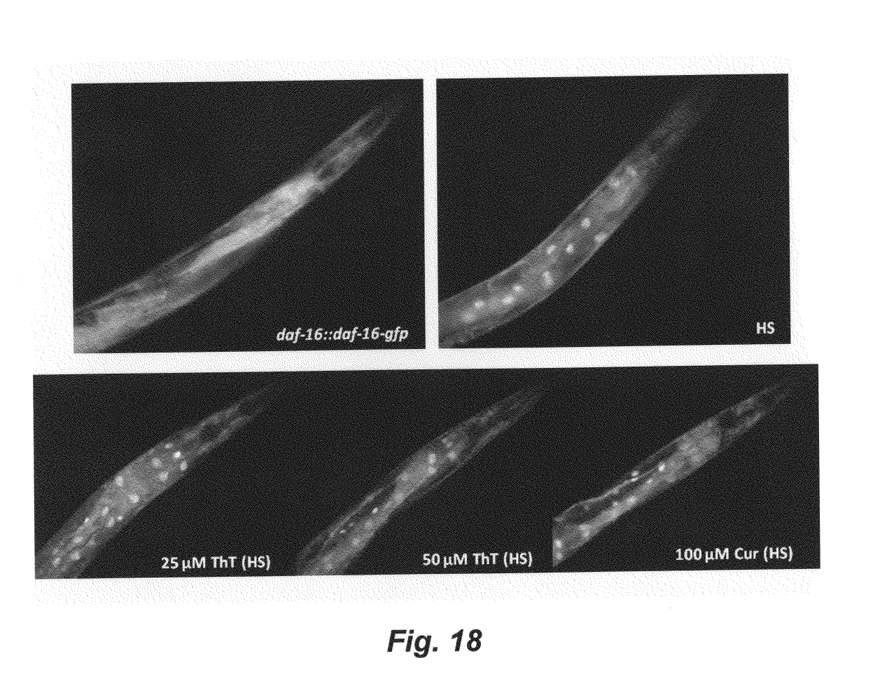

ThT treatment extended the lifespan of daf-16(mu86) worms lacking functional DAF-16 (FIG. 14b) and in a long-lived ILS mutant, age-1 (hx546), which is hypomorphic for the p110 catalytic subunit of a phosphoinositide 3-kinase (FIG. 17). In addition, ThT treatment did not alter the normal localization of a DAF-16 fusion protein [TJ356 daf-16::daf-16-gfp; pRF4 (rol-6(su1006))], unlike the nuclear relocalization observed in stressed worms (FIG. 14c; FIG. 18). ThT lifespan extension is therefore independent of ILS.

### Conclusion

In this study it was observed that compounds traditionally used to stain β-amyloid deposits confer a large increase in lifespan on C. elegans. The mechanism of ThT action was investigated, and ThT was found to suppress protein aggregation-associated paralysis in a range of toxic protein models in multiple tissues. ThT reduces Aβ₃₋₄₂ aggregation, decreases the levels of soluble aggregation-prone oligomeric proteins and localizes with these aggregates in vivo. The mechanism of aggregation suppression depends on molecular chaperones, autophagy and proteosomal functions. Finally, the extent of the ThT lifespan increase depends on the transcription factor HSF-1. The results indicate that ThT exerts its profound biological effect on longevity by slowing the formation of protein fibrils or aggregates. This may occur by the direct binding of ThT to intermediates in the pathways to a wide range of aggregates making them substrates for the proteostasis network. Additional mechanisms may be at play, such as activation of other detoxification systems. This modulation of proteostasis and protein aggregation pathways appears to have beneficial effects for healthspan and lifespan. These results suggest that small molecules targeting the protein homeostatic mechanisms provide opportunities for intervention in ageing and age-related disease.

### Materials and Methods

Nematode Growth and Strains

Strains were cultured under standard laboratory conditions. Strains used in this work include N2, HE250 [unc-52(e669su250)II], CB1157 [unc-54(e1157)I], CF1038 [daf-16(mu86)I], DA465 [eat-2(ad465)II], TJ1052 [age-1(hx546)II], PS3551 [hsf-1(sy441)I], TJ356 [zIs356 IV [daf-16::daf-16-gfp; pRF4 (rol-6(su1006))], CL 4176 [dvIs27(myo-3::Aβ(1 to 42)-let 3′UTR(pAF29); pRF4 (rol-6(su1006))], AM140 [rmIs132[P(unc-54) Q35::YFP], ZZ26 [unc-63(x26)I], CW152 [gas-1(fc21) X], CF2189 [Is001(skn-1::gfp+rol-6].

Lifespan Assay

Lifespan assays were performed as described previously. (McColl, G. et al. Pharmacogenetic analysis of lithium-induced delayed aging in Caenorhabditis elegans. J Biol Chem 283, 350-357 (2008). Briefly, the nematode growth media (NGM) plates were prepared under sterile conditions. 100 μL of concentrated stocks of each of the compounds used in this study were added onto a previously prepared NGM small plate (3 mL volume) immediately spread over the surface of the plate. The final concentrations quoted in the text assume an even distribution of compound throughout the 3 ml plate. The plates were then placed in a laminar flow hood at room temperature for 30 min and then 60 μL of a concentrated suspension of E. coli OP50 was spotted to form a circular lawn on the center of each plate. Thirty late L4 larvae growing at 20° C. were transferred to fresh NGM plates with FUdR (75 μM) in presence or absence of the specified compounds and incubated at 20° C. The first day of adulthood is Day 3 in survival curves. There was between-experiment variation in the magnitude of the lifespan extension observed with ThT which appeared to correlate with different suppliers and batches. ThT concentration should be optimized depending on batch and purity and stability of the compound. A darkening in the appearance of the stock resulted in loss of lifespan extension activity. The optimal range for lifespan extension was between 25 and 75 μM.

Animals were scored as alive, dead or lost every other day. Animals that failed to display touch-provoked movement were scored as dead. Animals that died from causes other than ageing, such as sticking to the plate walls, internal hatching of eggs (“bagging”) or gonadal extrusion were censored as were lost worms. Animals were transferred to fresh plates every 3-6 days. All lifespan experiments were performed at 20° C. unless otherwise stated. Survival curves were plotted and statistical analyses (log-rank test) were performed using the Prism 4 software (Graphpad™ Software, Inc., San Diego, Calif., USA).

Dietary Restriction

Plates were prepared as described (Chen, D., Thomas, E. L. & Kapahi, P. HIF-1 modulates dietary restriction-mediated lifespan extension via IRE-1 in Caenorhabditis elegans. PLoS Genet. 5, e1000486, (2009), but bacterial concentration was adjusted to 1.0×10¹² cfu/ml and diluted to achieve bacterial concentration of 1.0×10⁹ cfu/ml. Diluted bacterial cultures were spotted onto DR agar plates, which were modified from the standard nematode growth media (NGM) plates by excluding peptone and increasing agar from 1.7% to 2.0%. Carbenicillin (50 mg/ml) was added to the agar plates to further prevent bacterial growth. Synchronized L4 larvae grown under standard lab conditions (NGM plates with OP50 food, 20° C.) were transferred to fresh DR agar plates in presence or absence of 1, 10, 25, 50 and 100 μM ThT and lifespans scored as described above.

Demographic Analysis

Estimates of initial mortality rate and rate of increase with age and model fitting were made using WinModest. Gompertz mortality curves, ln(ux)=ln(a)+bx, where ux defines the age-specific hazard, were fitted with log-likelihood ratios used to examined the effects of constraining the intercept (a) or gradient (b) variables.

Worm Paralysis Assays

Populations of CL4176 dvIs2 [pCL12(unc-54/human Aβ₃₋₄₂ minigene)+pRF4J and AM140 rmIs1321P(unc-54) Q35::YFP] worms were grown at 20° C. for 48 h and then exposed to 50 μM ThT and 100 μM curcumin at 25° C. in presence of FUdR (50 μg/ml) for AM140. Scoring for paralysis was initiated 2 and 8 days after temperature upshift for CL4176 and AM140, respectively. Animals were scored as paralyzed if they failed to move during observation and exhibited “halos” of cleared bacteria around their heads (indicative of an insufficient body movement to access food), eggs accumulated close to the body or if the animals failed to respond to a touch-provoked movement with a platinum wire. For sensitivity to EtOH or levamisole resistance, CW152 gas-1 (fc21) X and ZZ26 unc-63(x26)I worms were picked into 0.4 M EtOH or 50 μM levamisole, respectively, equilibrated for 5 min, and scored for paralysis as described above. Treated and untreated worms were compared with an unpaired t-test (implemented in Prism 4, Graphpad™ Software, Inc., San Diego, Calif., USA).

Immunostaining and Photomicroscopv

For fluorescent microscopy, TJ356 zIs356 IV [daf-16::daf-16-gfp+rol-6] or CF2189 Is001(skn-1::gfp+rol-6) were paralyzed with 1 mM levamisole mounted on 1% agarose pads and imaged using Olympus BX51 (60× objective) and HCImage software (Hamamatsu). For immunofluorescence, N2, unc-He250 52(e669su250), CB1157 unc-54(e1157) or CL4176 dvIs27 [myo-3::Aβ(3 to 42)-let 3′UTR(pAF29); pRF4 (rol-6(su1006))] worms were treated for 24-36 h with or without 50 μM ThT at 25° C. After this period the worms were collected, rinsed, and fixed in 4% paraformaldehyde overnight. After fixation, worms were rinsed twice with 1 ml of 10 mM Tris-HCl pH 7.5 and then permeabilized by 24 h exposure to β-mercaptoethanol at 37° C. followed by collagenase treatment (2 mg/ml for 1-1.5 at 37° C.) to allow for digestion of the cuticle. Paramyosin and perlecans were detected with primary monoclonal antibodies 5-23 and MH3 (developed by Henry Epstein and Robert H. Waterston and obtained from the Developmental Studies Hybridoma Bank developed under the auspices of the NICHD and maintained by The University of Iowa, Department of Biology, Iowa City, Iowa 52242) and AlexaFluor 633 goat anti-mouse (Molecular Probes) as secondary antibody. Soluble oligomers and Aβ peptide were detected with anti polyclonal All (Invitrogen) and 6E10 monoclonal (Covance) primary antibodies, respectively, with AlexaFluor 568 goat anti-rabbit and AlexaFluor 488 goat anti-mouse (Molecular Probes) as secondary antibodies. Images were processed and quantified by using Image Analyst MK II (Novato, Calif.).

ThT distribution and potential colocalization with proteins prone to aggregate were explored by using two-photon excitation of ThT at 800 nm and emission at 435-485 nm in combination with anti oligomers and Aβ peptide immunodetection described above. In this spectral range worms exhibited negligible autofluorescence, therefore the signal was highly specific for ThT. Considering that image acquisition was performed after immunostaining likely only the protein-bound form of ThT was imaged.

Westernblot

Peptide corresponding to amino acids 110-145 (NLSEDGKLSIEAPKKEAVQGRSIPIQQAIVEEKSAE; SEQ ID NO:1) of HSP-16.2 was used to commercially synthesize antiserum (Invitrogen). Briefly, KLH-peptide was emulsified by mixing with an equal volume of Freund's adjuvant and injected into three subcutaneous dorsal sites for a total of 0.1 mg of peptide for immunization. The animals (rabbits) were bled, the blood allowed to clot and the serum collected by centrifugation. Monoclonal HSP-70 and Polyclonal HSF-1 primary antibodies were from stressgen (N27F3-4 and SPA-901, respectively).

For immunoblot analysis, 3-day-old adult hermaphrodites were treated with 50 μM ThT or 100 μM curcumin as described above and replicates of 25 animals were collected for each treatment. Worms were transferred to siliconized eppendorf tubes, washed once in S-basal and frozen in liquid N₂. Standard SDS-PAGE was performed using (4-12%) NOVEX gels and IVIES running buffer. Following transfer PVDF (BioRad) membranes were incubated with antisera (1:10000) or primary antibodies (1:1000) diluted in blocking buffer and then with secondary, goat anti-rabbit IgG antibody/horseradish peroxidase conjugate (Pierce), diluted 1:25000. Detection was undertaken with chemiluminescent reagents (SuperSignal, Pierce) and standard autoradiography.

RNA Interference Knockdown of Gene Expression

RNAi bacterial strains expressing double-stranded RNA that inactivates specified genes were cultured and utilized as previously described. (Timmons, L., Court, D. L. & Fire, A. Ingestion of bacterially expressed dsRNAs can produce specific and potent genetic interference in Caenorhabditis elegans. Gene 263, 103-112 (2001).) Briefly, eggs isolated from synchronous populations of unc-52(e669su250) cultures were placed on fresh RNAi plates and allowed to grow at 15° C.; 3 days later, L4 molt animals were transferred to new plates seeded with the same bacteria in presence or absence of ThT and switched to 25° C. The cultures were scored for paralysis after 48 h of treatment as described above. In all cases, 1 mM isopropyl-beta-D-thiogalactopyranoside (IPTG) was used for induction of double stranded RNA. In all the cases the identity of the clones was confirmed by sequencing.

Real Time Quantitative PCR Analysis

Twelve single adults from control or 50 μM ThT populations were picked after 3, 6 and 12 days of treatment into 5 μl of distilled water and flash frozen until extraction. Individual worms were extracted using the RNeasy Micro Kit, TissueLyser and QIAcube (Qiagen). Using the manufacturer's standard Animal Tissue protocol, each sample was homogenized in buffer RLT/Bme (Qiagen) on the TissueLyser (6 min total at 20 Hz). Samples, reagents and columns were then loaded into the QIAcube robot, and processed using the “RNeasy Micro—Animal tissues and cells—DNase digest” program. Each sample was then eluted in 14 μl of Nuclease-free water. cDNA templates for the Real Time PCR reactions were made using a combination of Message Sensor RT kit (Ambion's) and TaqMan PreAmp Master Mix (Applied Biosystems). The entire worm sample was added to the RT reaction and reversed transcribed at 50° C. for 20 min, using Message Sensor's “Two Step RT-PCR Protocol” (sample RT's were randomized). Then 2.5 μl of the cDNA sample was pre-amplified with a “4×Multiplex Primer Mix” for 14 cycles using TaqMan PreAmp master mix and protocol (ABI). Two modifications were made to ABI's protocol: 1. the reaction volume was reduced to 10 μl and 2. after pre-amplification, each cDNA sample was diluted 1:5 with 1XTE Buffer (Promega). cDNA samples were stored at −20° C. until needed. For identification of housekeeping genes, 6 potential housekeeping genes [act-2, act-3, gpd-1, gpd-2, gpd-4, rpb-2 and T19B4.3 (adenine phosphoribosyltransferase)] were profiled against 48 individual worms (8 worms from groups wildtype CTL and wildtype ThT at 3, 6 and 12 days of treatment) using the 48.48 Dynamic Array (BioMark) Real Time PCR Plate Set-Up and Protocol. From this experiment, 2 mRNAs (gpd-1 and gpd-4), were found to have invariant steady state levels across treatments and were used to derive Calibrated Normalized Relative Quantities (CNRQ) for each gene of interest. The mRNAs of interest, plus two housekeeping mRNAs, were run in triplicate against 89 single worm samples using the 96.96 Dynamic Array (BioMark) Real Time PCR Plate Set-Up and Protocol according to the manufacturer's protocol. Water runs were used as the blanks. mRNA transcript levels (CNQR) are plotted as arbitrary units (A.U.) Differences in relative mRNA transcript levels were identified using pair-wise t-tests.

### REFERENCES

1 Morimoto, R. I. Proteotoxic stress and inducible chaperone networks in neurodegenerative disease and aging. Genes Dev 22, 1427-1438 (2008).
2 Balch, W. E., Morimoto, R. I., Dillin, A. & Kelly, J. W. Adapting proteostasis for disease intervention. Science 319, 916-919 (2008).
3 Porat, Y., Abramowitz, A. & Gazit, E Inhibition of amyloid fibril formation by polyphenols: structural similarity and aromatic interactions as a common inhibition mechanism. Chem Biol Drug Des 67, 27-37 (2006).
4 Frid, P., Anisimov, S. V. & Popovic, N. Congo red and protein aggregation in neurodegenerative diseases. Brain Res Rev 53, 135-160 (2007).
5 Kenyon, C. The plasticity of aging: insights from long-lived mutants. Cell. 120, 449-460 (2005).
6 Tullet, J. M. et al. Direct inhibition of the longevity-promoting factor SKN-1 by insulin-like signaling in C. elegans. Cell 132, 1025-1038 (2008).
7 Morley, J. F. & Morimoto, R. I. Regulation of longevity in Caenorhabditis elegans by heat shock factor and molecular chaperones. Mol. Biol. Cell 15, 657-664 (2004).
8 Hsu, A. L., Murphy, C. T. & Kenyon, C. Regulation of aging and age-related disease by DAF-16 and heat-shock factor. Science 300, 1142-1145 (2003).
9 Cohen, E., Bieschke, J., Perciavalle, R. M., Kelly, J. W. & Dillin, A. Opposing activities protect against age-onset proteotoxicity. Science 313, 1604-1610 (2006).
10 Groenning, M. Binding mode of Thioflavin T and other molecular probes in the context of amyloid fibrils-current status. J Chem Biol (2009).
11 Drake, J., Link, C. D. & Butterfield, D. A. Oxidative stress precedes fibrillar deposition of Alzheimer's disease amyloid beta-peptide (1-42) in a transgenic Caenorhabditis elegans model. Neurobiology of aging 24, 415-420 (2003).
12 McColl, G. et al. THE Caenorhabditis elegans Abeta1-42 model of Alzheimer's disease predominantly expresses Abeta3-42. J Biol Chem (2009).
13 Temussi, P. A., Masino, L. & Pastore, A. From Alzheimer to Huntington: why is a structural understanding so difficult? EMBO J 22, 355-361 (2003).
14 Gidalevitz, T., Ben-Zvi, A., Ho, K. H., Brignull, H. R. & Morimoto, R. I. Progressive disruption of cellular protein folding in models of polyglutamine diseases. Science 311, 1471-1474 (2006).
15 Zengel, J. M. & Epstein, H. F. Identification of genetic elements associated with muscle structure in the nematode Caenorhabditis elegans. Cell motility 1, 73-97 (1980).
16 Anderson, P. & Brenner, S. A selection for myosin heavy chain mutants in the nematode Caenorhabditis elegans. Proc Natl Acad Sci USA 81, 4470-4474 (1984).
17 Ben-Zvi, A., Miller, E. A. & Morimoto, R. I. Collapse of proteostasis represents an early molecular event in Caenorhabditis elegans aging. Proc Natl Acad Sci USA 106, 14914-14919 (2009).
18 Lakowski, B. & Hekimi, S. The genetics of caloric restriction in Caenorhabditis elegans. Proc Natl Acad Sci USA 95, 13091-13096 (1998).
19 Chen, D., Thomas, E. L. & Kapahi, P. HIF-1 modulates dietary restriction-mediated lifespan extension via IRE-1 in Caenorhabditis elegans. PLoS Genet. 5, e1000486 (2009).
20 Murphy, C. T. et al. Genes that act downstream of DAF-16 to influence the lifespan of Caenorhabditis elegans. Nature 424, 277-283 (2003).
21 Walker, G. A. & Lithgow, G. J. Lifespan extension in C. elegans by a molecular chaperone dependent upon insulin-like signals. Aging cell 2, 131-139 (2003).
22 Li, W., Gao, B., Lee, S. M., Bennett, K. & Fang, D. RLE-1, an E3 ubiquitin ligase, regulates C. elegans aging by catalyzing DAF-16 polyubiquitination. Dev Cell 12, 235-246 (2007).
23 Ogg, S. et al. The Fork head transcription factor DAF-16 transduces insulin-like metabolic and longevity signals in C. elegans. Nature 389, 994-999 (1997).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entireties for all purposes.

1. A method of improving a measure of life span and/or health span, the method comprising administering an effective amount of a thioflavin compound, or derivative thereof, to a subject, whereby the measure of life span and/or health span is improved, and wherein the thioflavin compound has one of structures A-E: 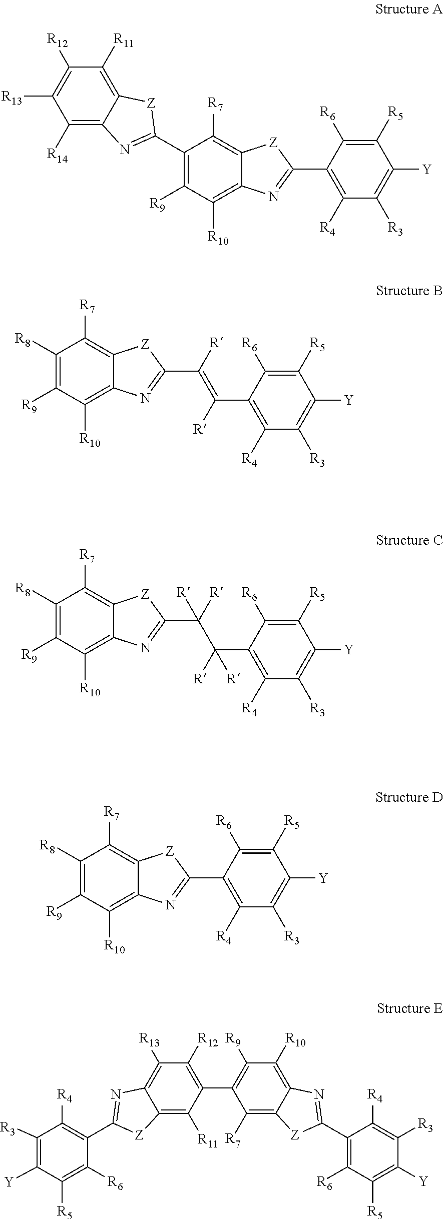
wherein Z is S, NR′, 0 or CR′ in which case the correct tautomeric form of the heterocyclic ring becomes an indole in which R′ is H or a lower alkyl group: 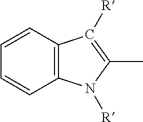
wherein Y is NR¹R², OR², or SR²;
wherein the nitrogen of 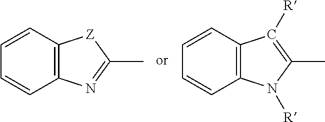
is not a quarternary amine;
or an thioflavin compound having one of structures F-J or a water soluble, non-toxic salt thereof: 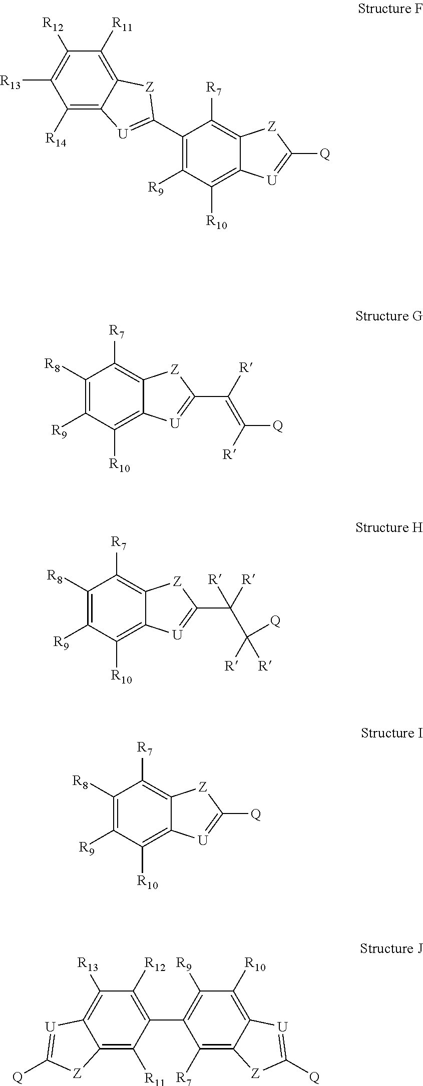
wherein each Q is independently selected from one of the following structures: 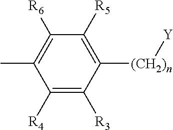
wherein n=0, 1, 2, 3 or 4, 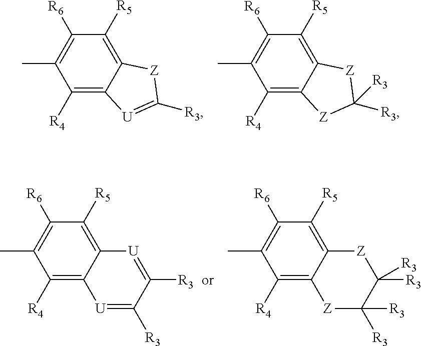
wherein Z is S, NR′, 0, or C(R′)₂ in which R′ is H or a lower alkyl group;
wherein U is CR′ (in which R′ is H or a lower alkyl group) or N (except when U═N, then Q is not 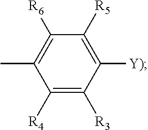
wherein Y is NR¹R², OR², or SR²;
wherein the nitrogen of 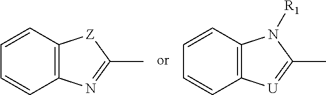
is not a quaternary amine;
wherein each R¹ and R² independently is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from any of the non-phenyl substituents defined below for R³-R¹⁴ and R′ is H or a lower alkyl group);
and wherein each R³-R¹⁴ independently is selected from the group consisting of H, F, Cl, Br, I, a lower alkyl group, (CH₂),OR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, N0₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from any of the non-phenyl substituents defined for R¹-R¹⁴ and wherein R′ is H or a lower alkyl group), a tri-alkyl tin and a chelating group (with or without a chelated metal group) of the form W-L or V—W-L, wherein V is selected from the group consisting of: —COO—, —CO—, —CH₂O— and —CH₂NH—; W is —(CH₂)ₙ where n=0, 1, 2, 3, 4, or 5; and L is: 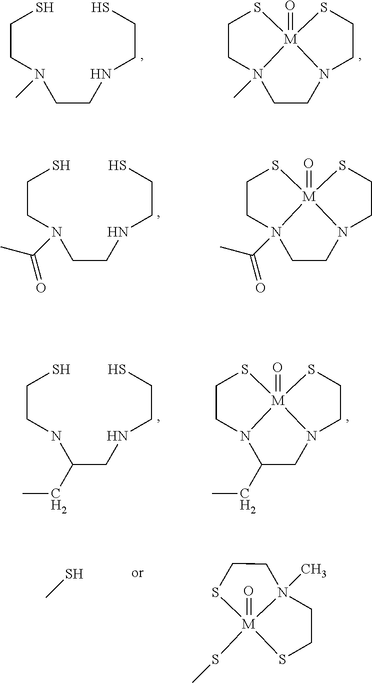
wherein M is selected from the group consisting of Tc, Re, Zn, Cu, Ni, V, Mn, Fe, Cr and Ru;
or wherein each R¹ and R² is a chelating group (with or without a chelated metal group) of the form W-L, wherein W is —(CH₂)ₙ where n=2, 3, 4, or 5; and L is: 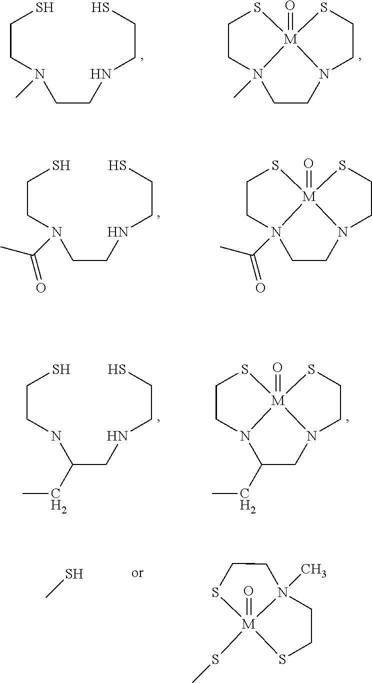
wherein M is selected from the group consisting of Tc and Re;
or wherein each R¹-R¹⁴ independently is selected from the group consisting of a chelating group (with or without a chelated metal ion) of the form W-L and V—W-L, wherein V is selected from the group consisting of —COO—, and —CO—; W is —(CH₂), where n=0, 1, 2, 3, 4, or 5; L is: 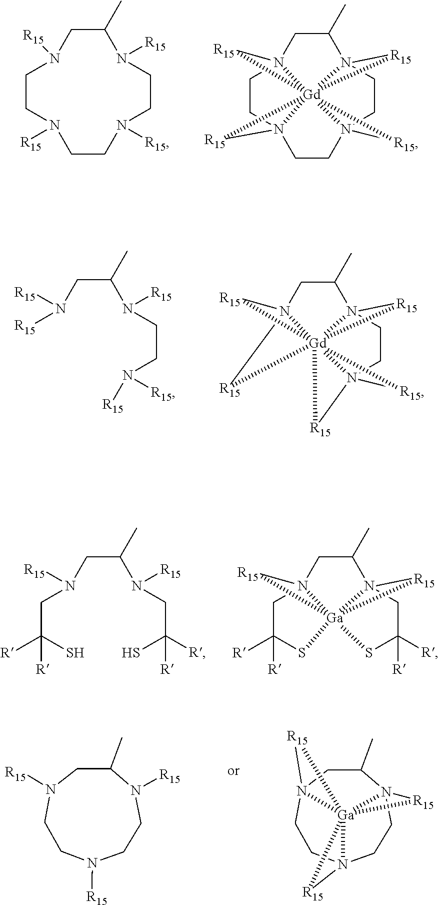
and wherein R¹⁵ independently is selected from the following: 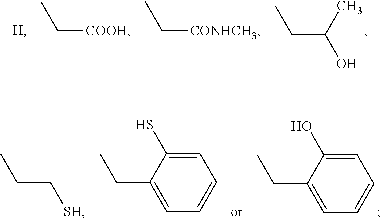
or a chelating compound (with or without a chelated metal group) or a water soluble, non-toxic salt thereof of the form: 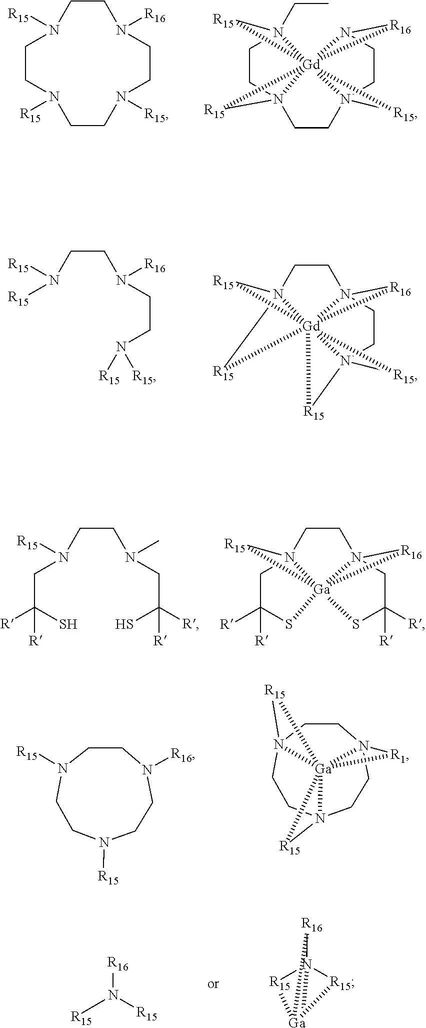
wherein R¹⁵ independently is selected from the following: 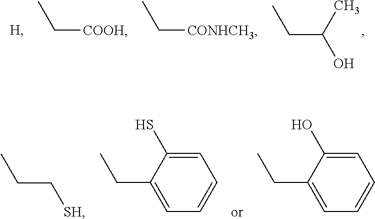
and R¹⁶ is 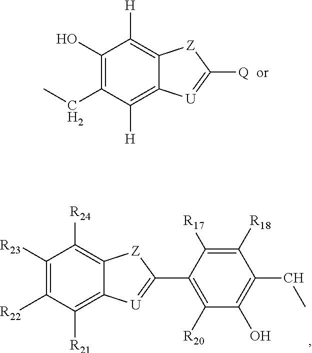
wherein
Q is independently selected from one of the following structures: 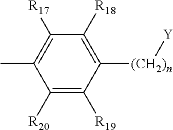
wherein n=0.1, 2, 3 or 4, 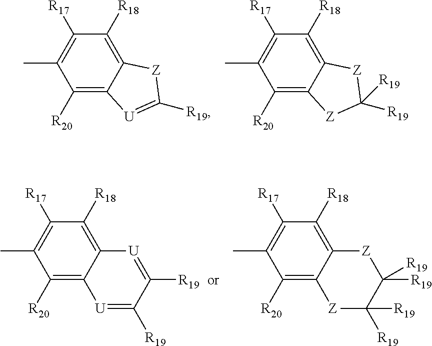
wherein Z is S, NR′, O, or C(R′)₂ in which R′ is H or a lower alkyl group;
wherein U is N or CR′;
wherein Y is NR¹R², OR², or SR²;
wherein each R¹⁷-R²⁴ independently is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ and CR₂′—CR₂′—Rₚₕ (wherein Rₚₕ represents an unsubstituted or substituted phenyl group with the phenyl substituents being chosen from any of the non-phenyl substituents defined for R¹⁷-R²⁰ and wherein R′ is H or a lower alkyl group).

2. The method of claim 1, wherein, Z═S, Y═N, R¹═H; and
wherein when the thioflavin compound of claim 1 is structure A or E, then R² is selected from the group consisting of a lower alkyl group, (CH₂)ₙOR′ (wherein n=1, 2, or 3), CF₃, CH₂—CH₂X, CH₂—CH₂—CH₂X (wherein X═F, Cl, Br or I), (C=0)-R′, Rph, and (CH₂)nRₚₕ wherein n=1, 2, 3, or 4;
wherein when the thioflavin compound of claim 1 is structure B, then R² is selected from the group consisting of (CH₂)ₙOR′ (wherein n=1, 2, or 3, and where when R′═H or CH₃, n is not 1), CF₃, CH₂—CH₂X and CH₂—CH₂—CH₂X (wherein X═F, Cl, Br or I);
wherein when the thioflavin compound of claim 1 is structure C, then R² is selected from the group consisting of a lower alkyl group, (CH₂)ₙOR′ (wherein n=1, 2, or 3, CF₃), CH₂—CH₂X, CH₂—CH₂—CH₂X (wherein X═F, Cl, Br or I), (C=0)-H, Rₚₕ, and (CH₂)ₙRₚₕ wherein n=1, 2, 3, or 4; and
wherein when the thioflavin compound of claim 1 is structure D, then R² is selected from the group consisting of (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4) wherein when R₂ is CH₂Rₚₕ R8 is not CH₃.

3-21. (canceled)

22. The method of claim 1, wherein the thioflavin compound comprises Thioflavin T (4-(3,6-dimethyl-1,3-benzothiazol-3-ium-2-yl)-N,N-dimethylaniline chloride (ThT)).

23. The method of claim 1, wherein the thioflavin compound does not comprise Thioflavin T (4-(3,6-dimethyl-1,3-benzothiazol-3-ium-2-yl)-N,N-dimethylaniline chloride (ThT).

24. A method of improving a measure of life span and/or health span, the method comprising administering to a subject an effective amount of one or more compounds selected from the group consisting of (2-(2-hydroxyphenyl)-benzoxazole (HBT), 2-(2-hydroxyphenyl)benzothiazole (HBX), 2-(2-aminophenyl)-1H-benzimidazole (BM), curcumin, and rifampicin and/or one or more derivatives thereof, whereby the measure of life span and/or health span is improved.

25. The method of claim 24, wherein the method comprises co-administering Thioflavin T (4-(3,6-dimethyl-1,3-benzothiazol-3-ium-2-yl)-N,N-dimethylaniline chloride (ThT) and curcumin.

26. The method of claim 1, wherein the improved measure of life span and/or health span comprises a reduction in frailty, an improvement in function in an age-related disability, the mitigation of a symptom of an age-related disease, and/or a delay in onset of frailty, age-related disability, or age-related disease, relative to the condition of the subject before administration of the compound or derivative or relative to a control population.

27. The method of claim 26, wherein the reduction in frailty is selected from the group consisting of increased strength, weight gain, faster mobility, increased energy, increased levels of activity, increased endurance, and enhanced behavioral response to a sensory cue, wherein the reduction is relative to the condition of the subject before administration of the compound or derivative or relative to a control population.

28. The method of claim 26, wherein the reduction in frailty is selected from the group consisting of a decrease in one or more inflammatory biomarkers, an improvement in glucose homeostasis, and a decrease in one of more biomarkers of clotting activation.

29. The method of claim 26, wherein the age-related disease is selected from the group consisting of osteoporosis, arthritis, cataracts, macular degeneration, and cardiovascular disease.

30. The method of claim 29, wherein the improved measure of life span and/or health span comprises an improvement in one or more parameters selected from the group consisting of cholesterol level, triglyceride level, high density lipoprotein level, and blood pressure.

31. The method of claim 1, wherein the improved measure of life span and/or health span comprises a reduction in, a reversal of, or delay in onset of sarcopenia, relative to the condition of the subject before administration of the compound or derivative or relative to a control population.

32. The method of claim 1, wherein the improved measure of life span and/or health span comprises a reduction in, a reversal of, or delay in onset of an age-related increase in lipofuscin accumulation in one or more tissues selected from the group consisting of brain, heart, liver, spleen, and kidney, relative to the condition of the subject before administration of the compound or derivative or relative to a control population.

33. The method of claim 26, wherein the subject is suffering from, or determined to be at risk for, frailty, an age-related disability, or an age-related disease.

34. The method of claim 33, wherein the subject is suffering from, or determined to be at risk for, frailty.

35. The method of claim 34, wherein the subject is determined to have at least three symptoms selected from the group consisting of weakness, weight loss, slowed mobility, fatigue, low levels of activity, poor endurance, and impaired behavioral response to a sensory cue.

36. The method of claim 34, wherein the subject is determined to have one or more symptoms selected from the group consisting of an increase in one or more inflammatory biomarkers, glucose homeostasis impairment, and an increase in one of more biomarkers of clotting activation.

37. The method of claim 33, wherein the subject is suffering from sarcopenia.

38. The method of claim 33, wherein the subject has lipofuscin accumulation in one or more tissues selected from the group consisting of brain, heart, liver, spleen, and kidney.

39. The method of claim 1, wherein the improvement in a measure of life span and/or health span comprises an enhanced ability to maintain homeostasis during the application of a stressor and/or a reduced time required to return to homeostasis after the application of a stressor.

40. (canceled)

41. The method of claim 39, wherein the subject has been determined to have a reduced ability to maintain homeostasis during the application of a stressor and/or an extended time required to return to homeostasis after the application of a stressor, wherein the reduced ability or extended time is relative to the condition of the subject at a previous time or relative to a normal ability or time.

42. The method of claim 1, wherein the measure of life span and/or health span comprises the level and/or activity of a molecule that plays a role in protein trafficking, the autophagy pathway, ubiquitination, and/or lysozomal degradation of proteins.

43. (canceled)

44. The method of claim 1, wherein the measure of life span and/or health span comprises the number of inclusion bodies in muscle tissue.

45. The method of claim 1, wherein the measure of life span and/or health span comprises mitochondrial function and/or morphology.

46. The method of claim 1, wherein the subject has been determined to have an abnormal level and/or activity of a molecule that plays a role in protein trafficking, the autophagy pathway, ubiquitination, and/or lysozomal degradation of proteins.

47. (canceled)

48. The method of claim 1, wherein the subject has been determined to have abnormal inclusion bodies in muscle tissue.

49. The method of claim 1, wherein the subject has been determined to have an abnormality in mitochondrial function and/or morphology.

50-52. (canceled)

53. The method of claim 1, wherein the improvement in the measure of life span and/or health span is at least about 40 percent, relative to the condition of the subject before administration of the compound or derivative or relative to a control population.

54-55. (canceled)

56. The method of claim 1, further comprising administering to said subject, an effective amount of an additional agent that is useful for increasing a measure of life span and/or health span.

57. The method of claim 56, wherein said additional agent is selected from the group consisting of a compound selected from the group consisting of an antioxidant, rapamycin, metformin, valproic acid, ethosuximide, trimethadione, 3,3-diethyl-2-pyrrolidinone, lithium, resveratrol, and derivatives thereof.
